(19) United States
(12) Patent Application Publication
(10) Pub. No.: US 2005/0142192 A1
(43) Pub. Date: Jun. 30, 2005

(54) Oral administration of [2-(8,9-dioxo-2,6-diazabicyclo[5.2.0]non-1(7)-en-2-yl)alkyl] phosphonic acid and derivatives

(75) Inventors: Eric J. Benjamin, Jamestown, NC (US); William F. Cloud, Garnerville, NY (US); Muhammad Ashraf, Elmwood Park, NJ (US); Mohammed Islam, Harriman, NY (US); Michael R. Brandt, Flemington, NJ (US); Gerald F. Tremblay, Lansdale, PA (US)

Correspondence Address: WOODCOCK WASHBURN LLP, ONE LIBERTY PLACE - 46TH FLOOR, PHILADELPHIA, PA, 19103, US

(73) Assignee: Wyeth, Madison, NJ (US)

(21) Appl. No.: 10/961,871

(22) Filed: Oct. 8, 2004

Related U.S. Application Data
(60) Provisional application No. 60/511,560, filed on Oct. 15, 2003.

(51) Int. Cl. A61K 31/675; A61K 9/20

(52) U.S. Cl. 424/464; 514/80

(57) ABSTRACT

Solid, pharmaceutical dosage forms of [2-(8,9-dioxo-2,6-diazabicyclo [5.2.0]non-1(7)-en-2-yl)alkyl]phosphonic acid and derivatives thereof are disclosed. In addition, methods of use are disclosed for the treatment, inter alia, of cerebral vascular disorders, anxiety disorders; mood disorders; schizophrenia; schizophreniform disorder; schizoaffective disorder; cognitive impairment; chronic neurodegenerative disorders; inflammatory diseases; fibromyalgia; complications from herpes zoster; prevention of tolerance to opiate analgesia; withdrawal symptoms from addictive drugs; and pain.

### CROSS REFERENCE TO RELATED APPLICATIONS

This application claims the benefit of U.S. Application No. 60/510,560 filed Oct. 15, 2003, the entire disclosure of which is incorporated herein by reference. This application is related to copending U.S. application Ser. No. 10/820,215, filed Apr. 7, 2004, and copending U.S. application Ser. No. 10/820,216, filed Apr. 7, 2004, the entire disclosures of which are incorporated herein by reference.

### FIELD OF THE INVENTION

The present invention relates to solid, pharmaceutical dosage forms of [2-(8,9-dioxo-2,6-diazabicyclo[5.2.0]non-1(7)-en-2-yl)alkyl]phosphonic acid and derivatives thereof, and methods of use thereof.

### BACKGROUND OF THE INVENTION

Substantial preclinical and clinical evidence indicates that inhibitors of the N-methyl-D-aspartate (NMDA) receptor have therapeutic potential for treating numerous disorders. Disorders believed to be responsive to inhibition of NMDA receptors include cerebral vascular disorders such as cerebral ischemia (e.g., stroke) or cerebral infarction resulting in a range of conditions, such as thromboembolic or hemorrhagic stroke, or cerebral vasospasm; cerebral trauma; muscular spasm; and convulsive disorders, such as epilepsy or status epilepticus. NMDA receptor antagonists may also be used to prevent tolerance to opiate analgesia or to help control symptoms of withdrawal from addictive drugs.

Screening of compounds in recent years have identified a number of NMDA receptor antagonists that have been used in animal and clinical human studies to demonstrate proof of concept for the treatment of a variety of disorders. The difficulty with demonstrating clinical utility of NMDA receptor antagonists has generally been the antagonists' lack of NMDA receptor subtype selectivity and/or biological activity when dosed orally.

[2-(8,9-Dioxo-2,6-diazabicyclo[5.2.0]non-1(7)-en-2-yl)alkyl]phosphonic acid and derivatives thereof have shown utility as NMDA receptor antagonists. See, for example, U.S. Pat. No. 5,168,103 and WO 03/031,416, the entire disclosures of which are herein incorporated by reference. The compound is very soluble in the pH range of 4 to 8. The apparent n-octanol/water partition coefficient is low (log partition coefficient is −1.37) and the Caco-2 cell permeability is poor, thus indicating low and incomplete oral absorption. Based on its high solubility and low permeability, [2-(8,9-dioxo-2,6-diazabicyclo[5.2.0]non-1(7)-en-2-yl)ethyl]phosphonic acid is classified as BCS Class 3. Animal absorption studies have shown that the compound has an oral bioavailability of approximately 1% at a dose of 100 mg/kg in rats and approximately 2.5% at a dose of 100 mg/kg in monkeys. Low bioavailabilities in this range have a potential of increasing the dose and the cost of the product. In addition, it may present problems of inter-subject plasma level variability in humans that may be further compounded by food-based absorption effects.

[2-(8,9-Dioxo-2,6-diazabicyclo[5.2.0]non-1(7)-en-2-yl)ethyl]phosphonic acid is a crystalline powder that has very low bulk density, poor flow and poor compressibility leading to problems in manufacturing capsules or tablets by dry blending processes, including blend segregation, poor content uniformity, and fill weight variation. Even the inclusion of directly compressible excipients may not solve these problems, especially when a large proportion, for example, greater than about 70%, by weight, based on the total weight of the formulation, of active pharmaceutical ingredient is desired in the formulation. Moreover, it is difficult by conventional dry blending methods to fill a formulation blend containing 300 mg of 2-(8,9-dioxo-2,6-diazabicyclo[5.2.0]non-1(7)-en-2-yl)alkyl]phosphonic acid or a derivative thereof without a densification step, because of the very low bulk density of the compound.

### SUMMARY OF THE INVENTION

The present invention provides pharmaceutical compositions and dosage forms comprising [2-(8,9-dioxo-2,6-diazabicyclo[5.2.0]non-1(7)-en-2-yl)alkyl] phosphonic acid or derivatives thereof. We have unexpectedly found that such compositions exhibit improved oral bioavailability.

In one embodiment, the invention is directed to solid, pharmaceutical dosage forms, comprising:
at least one compound of formula (I) or a pharmaceutically acceptable salt thereof: 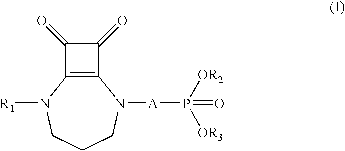
wherein:
R₁ is hydrogen, a C₁ to C₆ alkyl group, a C₂ to C₇ acyl group, a C₁ to C₆ alkanesulfonyl group, or a C₆ to C₁₄ aroyl group;
A is alkylenyl of 1 to 4 carbon atoms or alkenylenyl of 2 to 4 carbon atoms;
R₂ and R₃ are independently selected from hydrogen, 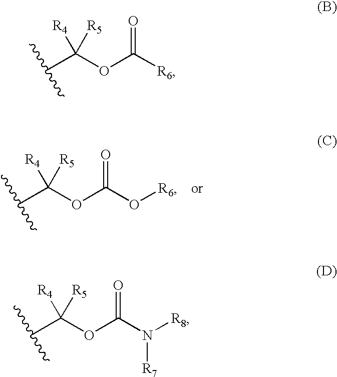
with the proviso that at least one of R₂ and R₃ is other than hydrogen;
R₄ and R₅ are independently selected from hydrogen, a C₁ to C₄ alkyl group, a C₅ to C₇ aryl group, a C₆ to C₁₅ aralkyl group having 5 to 7 carbon atoms in the aryl ring, a C₂ to C₇ alkenyl group, or C₂ to C₇ alkynyl group, or R₄ and R₅ may together form a spiro C₃ to C₈ carbocyclic ring;
R₆ is a C₁ to C₁₂ linear or branched alkyl group, a C₂ to C₇ linear or branched alkenyl or alkynyl group, a C₅ to C₁₃ aryl group, a C₆ to C₂₁ aralkyl group having 5 to 13 carbon atoms in the aryl moiety; a 5 to 13 membered heteroaryl group, a 6 to 21 membered heteroaralkyl group having 5 to 13 members in the heteroaryl moiety, a C₄ to C₈ cycloalkyl group, a C₅ to C₁₆ cycloalkylalkyl group having 4 to 8 carbon atoms in the cycloalkyl ring;
R₇ and R₈ are independently selected from hydrogen, a C₁ to C₁₂ linear or branched alkyl group, a C₂ to C₇ linear or branched alkenyl or alkynyl group, a C₅ to C₁₃ aryl group, a C₆ to C₂, aralkyl group having 5 to 13 carbon atoms in the aryl moiety, a 5 to 13 membered heteroaryl group, a 6- to 21-membered heteroaralkyl group having 5 to 13 members in the heteroaryl moiety, or R₇ and R₈ may together form a cycloalkyl or heterocycloalkyl group having in the ring 4 to 8 carbon atoms and optionally one to two atoms selected from nitrogen, oxygen or sulfur;
wherein any R₁ to R₈ group having an aryl, heteroaryl, cycloalkyl or heterocycloalkyl moiety may optionally be substituted on the aryl, heteroaryl, cycloalkyl or heterocycloalkyl moiety with 1 to about 5 substituents independently selected from a halo, a cyano, nitro or hydroxyl group, a C₁ to C₆ alkyl group, or a C₁ to C₆ alkoxy group; and
at least one pharmaceutically acceptable absorption enhancer.

In another embodiment, the invention is directed to solid, pharmaceutical dosage forms, comprising:
at least one compound of formula (I) or a pharmaceutically acceptable salt thereof: 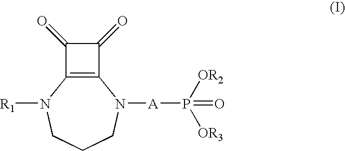
wherein:
R₁ is hydrogen;
A is —(CH₂)ₙ—, where n is 2; and
R₂ and R₃ are hydrogen; and
at least one pharmaceutically acceptable absorption enhancer.

In another embodiment, the invention is directed to methods for treating at least one condition in a mammal selected from a cerebral vascular disorder selected from cerebral ischemia, cerebral infarction or cerebral vasospasm; cerebral trauma; muscular spasm; a convulsive disorder selected from epilepsy or status epilepticus; hypoglycemia; cardiac arrest; asphyxia anoxia; or spinal chord injury, comprising the step of:
administering orally to a mammal in need thereof an effective amount of the solid, pharmaceutical dosage form described above.

In another embodiment, the invention is directed to methods for treating at least one condition in a mammal selected from glaucoma or diabetic end organ complications, comprising the step of:
administering orally to a mammal in need thereof an effective amount of the solid, pharmaceutical dosage form described above.

In another embodiment, the invention is directed to methods for treating at least one condition in a mammal selected from anxiety disorders; mood disorders; schizophrenia; schizophreniform disorder; or schizoaffective disorder, comprising the step of:
administering orally to a mammal in need thereof an effective amount of the solid, pharmaceutical dosage form described above.

In another embodiment, the invention is directed to methods for treating at least one neurodegenerative disorder in a mammal selected from Huntingdon's disease, Alzheimer's disease, amyotrophic lateral sclerosis, chronic dementia, or cognitive impairment, comprising the step of:
administering orally to a mammal in need thereof an effective amount of the solid, pharmaceutical dosage form described above.

In another embodiment, the invention is directed to methods for treating Parkinson's disease, comprising the step of:
administering orally to a mammal in need thereof an effective amount of the solid, pharmaceutical dosage form described above.

In another embodiment, the invention is directed to methods for treating at least one condition in a mammal selected from inflammatory diseases; fibromyalgia; complications from herpes zoster; prevention of tolerance to opiate analgesia; or withdrawal symptoms from addictive drugs, comprising the step of:
administering orally to a mammal in need thereof an effective amount of the solid, pharmaceutical dosage form described above.

In another embodiment, the invention is directed to methods for treating pain in a mammal, comprising the step of:
administering orally to a mammal in need thereof an effective amount of the solid, pharmaceutical dosage form described above.

In another embodiment, the invention is directed to solid, immediate release pharmaceutical compositions, comprising:
at least one compound of formula (I) or a pharmaceutically acceptable salt thereof: 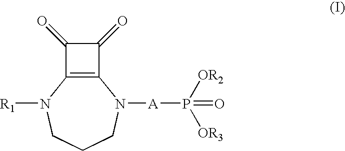
wherein:
R₁ is hydrogen, a C₁ to C₆ alkyl group, a C₂ to C₇ acyl group, a C₁ to C₆ alkanesulfonyl group, or a C₆ to C₁₄ aroyl group;
A is alkylenyl of 1 to 4 carbon atoms or alkenylenyl of 2 to 4 carbon atoms;
R₂ and R₃ are independently selected from hydrogen, 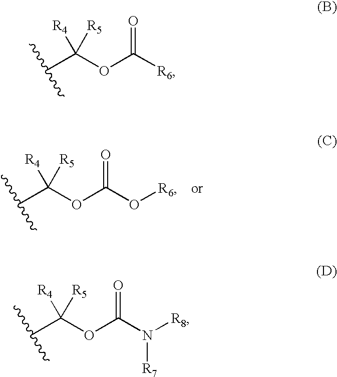
with the proviso that at least one of R₂ and R₃ is other than hydrogen;
R₄ and R₅ are independently selected from hydrogen, a C₁ to C₄ alkyl group, a C₅ to C₇ aryl group, a C₆ to C₁₅ aralkyl group having 5 to 7 carbon atoms in the aryl ring, a C₂ to C₇ alkenyl group, or C₂ to C₇ alkynyl group, or R₄ and R₅ may together form a spiro C₃ to C₈ carbocyclic ring;
R₆ is a C₁ to C₁₂ linear or branched alkyl group, a C₂ to C₇ linear or branched alkenyl or alkynyl group, a C₅ to C₁₃ aryl group, a C₆ to C₂, aralkyl group having 5 to 13 carbon atoms in the aryl moiety; a 5 to 13 membered heteroaryl group, a 6 to 21 membered heteroaralkyl group having 5 to 13 members in the heteroaryl moiety, a C₄ to C₈ cycloalkyl group, a C₅ to C₁₆ cycloalkylalkyl group having 4 to 8 carbon atoms in the cycloalkyl ring;
R₇ and R₈ are independently selected from hydrogen, a C₁ to C₁₂ linear or branched alkyl group, a C₂ to C₇ linear or branched alkenyl or alkynyl group, a C₅ to C₁₃ aryl group, a C₆ to C₂, aralkyl group having 5 to 13 carbon atoms in the aryl moiety, a 5 to 13 membered heteroaryl group, a 6- to 21-membered heteroaralkyl group having 5 to 13 members in the heteroaryl moiety, or R₇ and R₈ may together form a cycloalkyl or heterocycloalkyl group having in the ring 4 to 8 carbon atoms and optionally one to two atoms selected from nitrogen, oxygen or sulfur;
wherein any R₁ to R₈ group having an aryl, heteroaryl, cycloalkyl or heterocycloalkyl moiety may optionally be substituted on the aryl, heteroaryl, cycloalkyl or heterocycloalkyl moiety with 1 to about 5 substituents independently selected from a halo, a cyano, nitro or hydroxyl group, a C₁ to C₆ alkyl group, or a C₁ to C₆ alkoxy group;
wherein said composition has a bulk density of at least about 0.5 g/cm³.

In another embodiment, the invention is directed to solid, immediate release pharmaceutical compositions, comprising:
at least one compound of formula (I) or a pharmaceutically acceptable salt thereof: 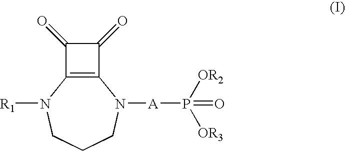
wherein:
R₁ is hydrogen;
A is —(CH₂)ₙ—, where n is 2; and
R₂ and R₃ are hydrogen; and
wherein said composition has a bulk density of at least about 0.5 g/cm³.

In other embodiments, the invention is directed to single dosage forms. In yet other embodiments, the invention is directed to multiple dosage forms.

In further embodiments, the invention is directed to capsules. In yet further embodiments, the invention is directed to tablets.

In another embodiment, the invention is directed to processes, comprising the steps of:
forming a wet granulation comprising:
   at least one binder;
   optionally at least one filler;
   optionally at least one disintegrant; and
   at least one compound of formula (I) or a pharmaceutically acceptable salt thereof; and
forming a solid dosage form.

In yet another embodiment, the invention is directed to products produced by the processes described above.

### BRIEF DESCRIPTION OF THE DRAWINGS

FIG. 1 is a plot of mean plasma concentration (in ng/mL) as a function of time (in hours) for a mean single dose of [2-(8,9-dioxo-2,6-diazabicyclo[5.2.0]non-1(7)-en-2-yl)ethyl]phosphonic acid in healthy subjects after receiving 200, 400, 800, or 1600 mg of [2-(8,9-dioxo-2,6-diazabicyclo[5.2.0]non-1(7)-en-2-yl)ethyl]phosphonic acid.

FIG. 2 is a plot of Cₘₐₓ (in ng/mL) as a function of dose (in mg) for a single dose of [2-(8,9-dioxo-2,6-diazabicyclo[5.2.0]non-1(7)-en-2-yl)ethyl]phosphonic acid in healthy subjects after receiving 200, 400, 800, or 1600 mg of [2-(8,9-dioxo-2,6-diazabicyclo[5.2.0]non-1(7)-en-2-yl)ethyl]phosphonic acid.

FIG. 3 is a plot of AUC (in ng·h/mL, t=0 to ∞) as a function of dose (in mg) for a single dose of [2-(8,9-dioxo-2,6-diazabicyclo[5.2.0]non-1(7)-en-2-yl)ethyl]phosphonic acid in healthy subjects after receiving 200, 400, 800, or 1600 mg of [2-(8,9-dioxo-2,6-diazabicyclo[5.2.0]non-1(7)-en-2-yl)ethyl]phosphonic acid.

FIG. 4 is a plot of mean steady state plasma concentration (in ng/mL) as a function of time (in hours) for [2-(8,9-dioxo-2,6-diazabicyclo[5.2.0]non-1(7)-en-2-yl)ethyl]phosphonic acid in healthy subjects after receiving 200, 400, 800, or 1600 mg of [2-(8,9-dioxo-2,6-diazabicyclo[5.2.0]non-1(7)-en-2-yl)ethyl]phosphonic acid.

FIG. 5 is a plot of steady state Cₘₐₓ (in ng/mL) as a function of dose (in mg) for [2-(8,9-dioxo-2,6-diazabicyclo[5.2.0]non-1(7)-en-2-yl)ethyl]phosphonic acid in healthy subjects after receiving 200, 400, 800, or 1600 mg of [2-(8,9-dioxo-2,6-diazabicyclo[5.2.0]non-1(7)-en-2-yl)ethyl]phosphonic acid.

FIG. 6 is a plot of steady state AUC (in ng·h/mL, t=0 to tau (12 hours)) as a function of dose (in mg) for [2-(8,9-dioxo-2,6-diazabicyclo[5.2.0]non-1(7)-en-2-yl)ethyl]phosphonic acid in healthy subjects after receiving 200, 400, 800, or 1600 mg of [2-(8,9-dioxo-2,6-diazabicyclo[5.2.0]non-1(7)-en-2-yl)ethyl]phosphonic acid.

### DETAILED DESCRIPTION OF THE INVENTION

As employed above and throughout the disclosure, the following terms, unless otherwise indicated, shall be understood to have the following meanings.

“Alkyl,” as used herein, refers to an aliphatic hydrocarbon radical having 1 to 12 carbon atoms and includes, but is not limited to, straight or branched chains such as methyl, ethyl, n-propyl, isopropyl, n-butyl, isobutyl, sec-butyl, t-butyl, n-pentyl, isopentyl, neo-pentyl, n-hexyl, and isohexyl. “Lower alkyl” refers to alkyl having 1 to 3 carbon atoms.

“Alkylenyl,” as used herein, refers to an aliphatic hydrocarbon diradical having 1 to 12 carbon atoms and includes, but is not limited to, straight or branched chains such as methylenyl, ethylenyl, n-propylenyl, isopropylenyl, n-butylenyl, isobutylenyl, sec-butylenyl, t-butylenyl, n-pentylenyl, isopentylenyl, neo-pentylenyl, n-hexylenyl, and isohexylenyl. “Lower alkylenyl” refers to alkylenyl having 1 to 3 carbon atoms.

“Alkenyl,” as used herein, refers to an aliphatic straight or branched hydrocarbon radical having 2 to 7 carbon atoms that may contain 1 to 3 double bonds. Examples of alkenyl are straight or branched mono-, di-, or polyunsaturated groups such as vinyl, prop-1-enyl, allyl, methallyl, but-1-enyl, but-2-enyl and but-3-enyl.

“Alkenylenyl,” as used herein, refers to an aliphatic straight or branched hydrocarbon diradical having 2 to 7 carbon atoms that may contain 1 to 3 double bonds. Examples of alkenylenyl are straight or branched mono-, di-, or polyunsaturated groups such as vinylenyl, prop-1-enylenyl, allylenyl, methallylenyl, but-1-enylenyl, but-2-enylenyl and but-3-enylenyl.

“Alkynyl,” as used herein, refers to an aliphatic, straight or branched, hydrocarbon chain having 2 to 7 carbon atoms that may contain 1 to 3 triple bonds.

“Acyl,” as used herein, refers to the group R—C(═O)— where R is an alkyl group of 1 to 5 carbon atoms. For example, a C₂ to C₇ acyl group refers to the group R—C(═O)— where R is an alkyl group of 1 to 6 carbon atoms.

“Alkanesulfonyl,” as used herein, refers to the group R—S(O)₂— where R is an alkyl group of 1 to 6 carbon atoms.

“Aryl,” as used herein, refers to an aromatic 5- to 13-membered mono- or bi-carbocyclic ring such as phenyl or naphthyl. Preferably, groups containing aryl moieties are monocyclic having 5 to 7 carbon atoms in the ring. “Heteroaryl,” (Het Ar), as used herein, means an aromatic 5- to 13-membered carbon containing mono- or bi-cyclic ring having one to five heteroatoms that independently may be nitrogen, oxygen or sulfur. Preferably, groups containing heteroaryl moieties are monocyclic having 5 to 7 members in the ring where one to two of the ring members are selected independently from nitrogen, oxygen or sulfur. Groups containing aryl or heteroaryl moieties may optionally be substituted as defined below or unsubstituted.

“Aroyl,” as used herein, refers to the group Ar—C(═O)— where Ar is aryl as defined above. For example, a C₆ to C₁₄ aroyl moiety refers to the group Ar—C(═O)— where Ar is an aromatic 5 to 13 membered carbocylic ring.

“Aralkyl,” as used herein, refers to the group —R—Ar where Ar is aryl as defined above and R is an alkylene moiety having 1 to 8, preferably 1 to 6, and more preferably 1 to 4 carbon atoms. Examples of aralkyl groups include benzyl, phenethyl, 3-phenylpropyl, and 4-phenyl butyl.

“Heteroaralkyl,” as used herein refers to the group —R-hetAr where hetAr is heteroaryl as defined above and R is an alkylene moiety having 1 to 8, preferably 1 to 6, and more preferably 1 to 4 carbon atoms.

“Cycloalkyl,” as used herein refers to a monocarbocyclic ring having 3 to 8 carbon atoms. Groups containing cycloalkyl may optionally be substituted as defined below or unsubstituted.

“Heterocycloalkyl,” as used herein, refers to a carbon containing monocyclic ring having 3 to 8 ring members where one to two ring atoms are independently selected from nitrogen, oxygen or sulfur. Groups containing heterocycloalkyl moieties may optionally be substituted as defined below or unsubstituted.

“Cycloalkylalkyl,” as used herein, refers to the group —R-cycloalk where cycloalk is a cycloalkyl group as defined above and R is an alkylene moiety having 1 to 8, preferably 1 to 6, and more preferably 1 to 4 carbon atoms.

“Halo,” as used herein, means fluoro, chloro, bromo or iodo.

“Pharmaceutically acceptable,” as used herein, means a substance that is acceptable for use in pharmaceutical applications from a toxicological perspective and does not adversely interact with the active ingredient.

“Substituted,” as used herein, refers to a moiety, such as an aryl, heteroaryl, cycloalkyl or heterocycloalkyl moiety having from 1 to about 5 substituents, and more preferably from 1 to about 3 substituents independently selected from a halo, a cyano, nitro or hydroxyl group, a C₁ to C₆ alkyl group, or a C₁ to C₆ alkoxy group. Preferred substituents are a halo, a hydroxyl group, or a C₁ to C₆ alkyl group.

“Cₘₐₓ,” “Tₘₐₓ,” and “AUC” values reported herein, unless stated as being “mean” values, refer to the values observed in an individual patient. Moreover, Cₘₐₓ, Tₘₐₓ, and AUC values, unless otherwise stated, may be values observed at steady state when dosing at regular time intervals (e.g., every 12 hours) for multiple days (e.g., multiple dose administration) or values for a single dose administration.

Accordingly, in one embodiment, the present invention provides solid, pharmaceutical dosage forms, comprising:
at least one compound of formula (I) or a pharmaceutically acceptable salt thereof: 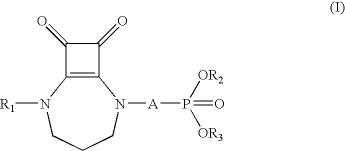
wherein:
R₁ is hydrogen, a C₁ to C₆ alkyl group, a C₂ to C₇ acyl group, a C₁ to C₆ alkanesulfonyl group, or a C₆ to C₁₄ aroyl group;
A is alkylenyl of 1 to 4 carbon atoms or alkenylenyl of 2 to 4 carbon atoms;
R₂ and R₃ are independently selected from hydrogen, 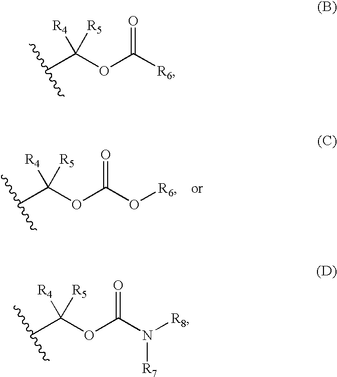
with the proviso that at least one of R₂ and R₃ is other than hydrogen;
R₄ and R₅ are independently selected from hydrogen, a C₁ to C₄ alkyl group, a C₅ to C₇ aryl group, a C₆ to C₁₅ aralkyl group having 5 to 7 carbon atoms in the aryl ring, a C₂ to C₇ alkenyl group, or C₂ to C₇ alkynyl group, or R₄ and R₅ may together form a spiro C₃ to C₈ carbocyclic ring;
R₆ is a C₁ to C₁₂ linear or branched alkyl group, a C₂ to C₇ linear or branched alkenyl or alkynyl group, a C₅ to C₁₃ aryl group, a C₆ to C₂, aralkyl group having 5 to 13 carbon atoms in the aryl moiety; a 5 to 13 membered heteroaryl group, a 6 to 21 membered heteroaralkyl group having 5 to 13 members in the heteroaryl moiety, a C₄ to C₈ cycloalkyl group, a C₅ to C₁₆ cycloalkylalkyl group having 4 to 8 carbon atoms in the cycloalkyl ring;
R₇ and R₈ are independently selected from hydrogen, a C₁ to C₁₂ linear or branched alkyl group, a C₂ to C₇ linear or branched alkenyl or alkynyl group, a C₅ to C₁₃ aryl group, a C₆ to C₂, aralkyl group having 5 to 13 carbon atoms in the aryl moiety, a 5 to 13 membered heteroaryl group, a 6- to 21-membered heteroaralkyl group having 5 to 13 members in the heteroaryl moiety, or R₇ and R₈ may together form a cycloalkyl or heterocycloalkyl group having in the ring 4 to 8 carbon atoms and optionally one to two atoms selected from nitrogen, oxygen or sulfur;
wherein any R₁ to R₈ group having an aryl, heteroaryl, cycloalkyl or heterocycloalkyl moiety may optionally be substituted on the aryl, heteroaryl, cycloalkyl or heterocycloalkyl moiety with 1 to about 5 substituents independently selected from a halo, a cyano, nitro or hydroxyl group, a C₁ to C₆ alkyl group, or a C₁ to C₆ alkoxy group; and
at least one pharmaceutically acceptable absorption enhancer.

In another embodiment, the invention is directed to solid, pharmaceutical dosage forms, comprising:
at least one compound of formula (I) or a pharmaceutically acceptable salt thereof: 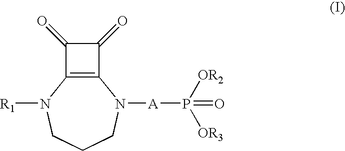
wherein:
R₁ is hydrogen;
A is —(CH₂)ₙ—, where n is 2; and
R₂ and R₃ are hydrogen; and
at least one pharmaceutically acceptable absorption enhancer.

In another embodiment, the invention is directed to solid, immediate release pharmaceutical compositions, comprising:
at least one compound of formula (I) or a pharmaceutically acceptable salt thereof: 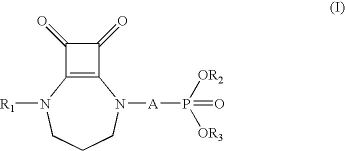
wherein:
R₁ is hydrogen, a C₁ to C₆ alkyl group, a C₂ to C₇ acyl group, a C₁ to C₆ alkanesulfonyl group, or a C₆ to C₁₄ aroyl group;
A is alkylenyl of 1 to 4 carbon atoms or alkenylenyl of 2 to 4 carbon atoms;
R₂ and R₃ are independently selected from hydrogen, 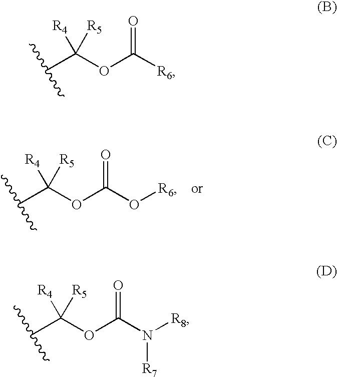
with the proviso that at least one of R₂ and R₃ is other than hydrogen;
R₄ and R₅ are independently selected from hydrogen, a C₁ to C₄ alkyl group, a C₅ to C₇ aryl group, a C₆ to C₁₅ aralkyl group having 5 to 7 carbon atoms in the aryl ring, a C₂ to C₇ alkenyl group, or C₂ to C₇ alkynyl group, or R₄ and R₅ may together form a spiro C₃ to C₈ carbocyclic ring;
R₆ is a C₁ to C₁₂ linear or branched alkyl group, a C₂ to C₇ linear or branched alkenyl or alkynyl group, a C₅ to C₁₃ aryl group, a C₆ to C₂, aralkyl group having 5 to 13 carbon atoms in the aryl moiety; a 5 to 13 membered heteroaryl group, a 6 to 21 membered heteroaralkyl group having 5 to 13 members in the heteroaryl moiety, a C₄ to C₈ cycloalkyl group, a C₅ to C₁₆ cycloalkylalkyl group having 4 to 8 carbon atoms in the cycloalkyl ring;
R₇ and R₈ are independently selected from hydrogen, a C₁ to C₁₂ linear or branched alkyl group, a C₂ to C₇ linear or branched alkenyl or alkynyl group, a C₅ to C₁₃ aryl group, a C₆ to C₂₁ aralkyl group having 5 to 13 carbon atoms in the aryl moiety, a 5 to 13 membered heteroaryl group, a 6- to 21-membered heteroaralkyl group having 5 to 13 members in the heteroaryl moiety, or R₇ and R₈ may together form a cycloalkyl or heterocycloalkyl group having in the ring 4 to 8 carbon atoms and optionally one to two atoms selected from nitrogen, oxygen or sulfur;
wherein any R₁ to R₈ group having an aryl, heteroaryl, cycloalkyl or heterocycloalkyl moiety may optionally be substituted on the aryl, heteroaryl, cycloalkyl or heterocycloalkyl moiety with 1 to about 5 substituents independently selected from a halo, a cyano, nitro or hydroxyl group, a C₁ to C₆ alkyl group, or a C₁ to C₆ alkoxy group;
wherein said composition has a bulk density of at least about 0.5 g/cm³, preferably at least about 0.8 g/cm³.

In another embodiment, the invention is directed to solid, immediate release pharmaceutical compositions, comprising:
at least one compound of formula (I) or a pharmaceutically acceptable salt thereof: 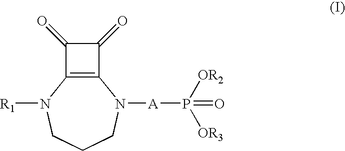
wherein:
R₁ is hydrogen;
A is —(CH₂)ₙ—, where n is 2; and
R₂ and R₃ are hydrogen; and
wherein said composition has a bulk density of at least about 0.5 g/cm³, preferably at least about 0.8 g/cm³.

In other embodiments, the invention is directed to single dosage forms. In yet other embodiments, the invention is directed to multiple dosage forms.

In further embodiments, the invention is directed to capsules. In yet further embodiments, the invention is directed to tablets.

In certain embodiments, the invention is directed to solid, immediate release pharmaceutical compositions, wherein the composition exhibits a plasma Cₘₐₓ, upon administration to a subject in need thereof, for the compound of formula (I) of about 80 ng/mL to about 4200 ng/mL, preferably at least about 200 ng/mL, more preferably at least about 270 ng/mL, even more preferably at least about 2940 ng/mL.

In certain embodiments, the invention is directed to solid, immediate release pharmaceutical compositions,
wherein the composition exhibits a plasma Tₘₐₓ, upon administration to a subject in need thereof, for the compound of formula (I) of about 0.5 hours to about 4.0 hours.

In certain embodiments, the invention is directed to solid, immediate release pharmaceutical compositions,
wherein the composition exhibits an AUC (t=0 to 12 hours) upon administration to a subject in need thereof, for the compound of formula (I) of about 250 ng·h/mL to about 6,000 ng·h/mL, preferably at least about 510 ng·h/mL, more preferably at least about 1215 ng·h/mL, even more preferably at least about 1280 ng·h/mL, and yet even more preferably at least about 2850 ng·h/mL. In a preferred embodiment, when the composition is dosed at a regular interval (e.g., every 12 hours) for one or more days, the AUC total daily exposure (t=0 to 24 hours) is from about 500 ng·h/mL to about 12,000 ng·h/mL, preferably at least about 1020 ng·h/mL, more preferably at least about 2430 ng·h/mL, even more preferably 2560 ng·h/mL, and yet even more preferably at least about 5700 ng·h/mL.

The solid, pharmaceutical dosage form of the present invention may be in any suitable solid dosage form for oral administration. Examples of suitable solid dosage forms include powders, capsules, tablets, pills, troches, cachets and pellets. Preferably, the solid dosage form for oral administration is a capsule or a tablet. The dosage forms may be enteric-coated or prepared for immediate release. In preferred embodiments, the capsule or tablet is enteric-coated.

The capsule material may be either hard or soft, and as will be appreciated by those skilled in the art, typically comprises a tasteless, easily administered and water soluble compound, such as gelatin, starch or a cellulosic material. The capsules are preferably sealed, such as with gelatin bands or the like. See, for example, Remington: The Science and Practice of Pharmacy, 20^{th} Edition (Easton, Pa.: Mack Publishing Company, 2000), which describes materials and methods for preparing encapsulated pharmaceuticals.

The enteric coating is typically, although not necessarily, a polymeric material. Preferred enteric coating materials comprise bioerodible, gradually hydrolyzable and/or gradually water-soluble polymers. The “coating weight,” or relative amount of coating material per capsule, generally dictates the time interval between ingestion and drug release. Any coating should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the practice of the present invention to achieve delivery of the active to the lower gastrointestinal tract. The selection of the specific enteric coating material will depend on the following properties: resistance to dissolution and disintegration in the stomach; impermeability to gastric fluids and drug/carrier/enzyme while in the stomach; ability to dissolve or disintegrate rapidly at the target intestine site; physical and chemical stability during storage; non-toxicity; ease of application as a coating (substrate friendly); and economical practicality.

Suitable enteric coating materials include, but are not limited to: cellulosic polymers, such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, preferably formed from acrylic acid, met acrylic acid, methyl acrylate, ammonium methylacrylate, ethyl acrylate, methyl methacrylate and/or ethyl methacrylate (e.g., those copolymers sold under the trade name EUDRAGIT); vinyl polymers and copolymers, such as polyvinyl pyrrolidone (PVP), polyvinyl acetate, polyvinyl acetate phthalate, vinyl acetate crotonic acid copolymer, and ethylene-vinyl acetate copolymers; and shellac (purified lac). Combinations of different coating materials may also be used to coat a single capsule.

The enteric coating provides for controlled release of the active agent, such that drug release can be accomplished at some generally predictable location in the lower intestinal tract below the point at which drug release would occur without the enteric coating. The enteric coating also prevents exposure of the hydrophilic therapeutic agent and carrier to the epithelial and mucosal tissue of the buccal cavity, pharynx, esophagus, and stomach, and to the enzymes associated with these tissues. The enteric coating therefore helps to protect the active agent and a patient's internal tissue from any adverse event prior to drug release at the desired site of delivery. Furthermore, the coated capsules of the present invention allow optimization of drug absorption, active agent protection, and safety. Multiple enteric coatings targeted to release the active agent at various regions in the lower gastrointestinal tract would enable even more effective and sustained improved delivery throughout the lower gastrointestinal tract.

The coating may, and preferably does, contain a plasticizer to prevent the formation of pores and cracks that would permit the penetration of the gastric fluids. Suitable plasticizers include, but are not limited to, triethyl citrate (CITROFLEX 2), triacetin (glyceryl triacetate), acetyl triethyl citrate (CITROFLEC A2), CARBOWAX 400 (polyethylene glycol 400), diethyl phthalate, tributyl citrate, acetylated monoglycerides, glycerol, fatty acid esters, propylene glycol, and dibutyl phthalate. In particular, a coating comprised of an anionic carboxylic acrylic polymer will typically contain less than about 50% by weight, preferably less than about 30% by weight, and more preferably, about 10% to about 25% by weight, based on the total weight of the coating, of a plasticizer, particularly dibutyl phthalate, polyethylene glycol, triethyl citrate and triacetin. The coating may also contain other coating excipients, such as detackifiers, antifoaming agents, lubricants (e.g., magnesium stearate), and stabilizers (e.g., hydroxypropylcellulose, acids and bases) to solubilize or disperse the coating material, and to improve coating performance and the coated product.

In preferred embodiments, the enteric-coated capsule or the enteric-coated tablet comprises a coating formed from an anionic polymer selected from the group consisting of a methacrylic acid copolymer, cellulose acetate phthalate, hydroxpropylmethylcellulose phthalate, polyvinyl acetate phthalate, shellac, hydroxpropylmethylcellulose acetate succinate, and carboxy-methylcellulose. In particularly preferred embodiments, the enteric coating is a methacrylic acid copolymer.

The coating may be applied to the capsule or tablet using conventional coating methods and equipment. For example, an enteric coating may be applied to a capsule using a coating pan, an airless spray technique, fluidized bed coating equipment, or the like. Detailed information concerning materials, equipment and processes for preparing coated dosage forms may be found in Pharmaceutical Dosage Forms: Tablets, eds. Lieberman et al. (New York: Marcel Dekker, Inc., 1989), and in Ansel et al., Pharmaceutical Dosage Forms and Drug Delivery Systems, 6^{th} Edition (Media, Pa.: Williams & Wilkins, 1995). The coating thickness, as noted above, must be sufficient to ensure that the oral dosage form remains intact until the desired site of topical delivery in the lower intestinal tract is reached.

In another embodiment of the present invention, the solid, pharmaceutical dosage form is in unit dosage or multiple dose form. In such form, the composition is sub-divided in unit or multiple doses containing appropriate quantities of the active ingredient. The dosage forms can be packaged compositions. Thus, the present invention also provides a solid, pharmaceutical dosage form in unit dosage or multiple dose form containing an effective unit or multiple dosage for oral administration of at least one compound of formula (I) or a pharmaceutically acceptable salt thereof; at least one pharmaceutically acceptable absorption enhancer; and optionally, at least one additive for forming a solid dosage form.

As one skilled in the art will recognize, the preferred effective unit or multiple dosage will depend on for example, the condition being treated and the particular compound chosen for formula I. For example, it is believed that compounds of formula (I) where R₂ and/or R₃ are the moiety B, C, or D may have improved bioavailability, and may thus be dosed at lower dosages, relative to compounds of formula (I) where R₂ and R₃ are hydrogen. Preferably, however, a dosage (whether in unit or multiple dosage form) for daily oral administration will range from about 400 mg (200 mg BID) to about 4000 mg (2000 mg BID) and more preferably from about 400 mg (200 mg BID) to about 3200 mg (1600 mg BID) of the compound of formula (I) useful in the present invention. In certain embodiments, a daily dosage (whether in unit or multiple dosage form) for oral administration will range from about 800 mg (400 mg BID) to about 3200 mg (1600 mg BID) and more preferably from about 800 mg (400 mg BID) to about 1200 mg (600 mg BID) of the compound of formula (I) useful in the present invention.

In formula (I) above:
R₁ is hydrogen, a C₁ to C₆ alkyl group, a C₂ to C₇ acyl group, a C₁ to C₆ alkanesulfonyl group, or a C₆ to C₁₄ aroyl group;
A is alkylenyl of 1 to 4 carbon atoms or alkenylenyl of 2 to 4 carbon atoms;
R₂ and R₃ are independently selected from hydrogen, 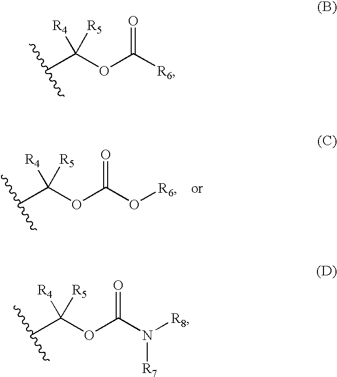
R₄ and R₅ are independently selected from hydrogen, a C₁ to C₄ alkyl group, a C₅ to C₇ aryl group, a C₆ to C₁₅ aralkyl group having 5 to 7 carbon atoms in the aryl ring, a C₂ to C₇ alkenyl group, or C₂ to C₇ alkynyl group, or R₄ and R₅ may together form a spiro C₃ to C₈ carbocyclic ring;
R₆ is a C₁ to C₁₂ linear or branched alkyl group, a C₂ to C₇ linear or branched alkenyl or alkynyl group, a C₅ to C₁₃ aryl group, a C₆ to C₂, aralkyl group having 5 to 13 carbon atoms in the aryl moiety; a 5 to 13 membered heteroaryl group, a 6 to 21 membered heteroaralkyl group having 5 to 13 members in the heteroaryl moiety, a C₄ to C₈ cycloalkyl group, a C₅ to C₁₆ cycloalkylalkyl group having 4 to 8 carbon atoms in the cycloalkyl ring;
R₇ and R₈ are independently selected from hydrogen, a C₁ to C₁₂ linear or branched alkyl group, a C₂ to C₇ linear or branched alkenyl or alkynyl group, a C₅ to C₁₃ aryl group, a C₆ to C₂, aralkyl group having 5 to 13 carbon atoms in the aryl moiety, a 5 to 13 membered heteroaryl group, a 6- to 21-membered heteroaralkyl group having 5 to 13 members in the heteroaryl moiety, or R₇ and R₈ may together form a cycloalkyl or heterocycloalkyl group having in the ring 4 to 8 carbon atoms and optionally one to two atoms selected from nitrogen, oxygen or sulfur;
wherein any R₁ to R₈ group having an aryl, heteroaryl, cycloalkyl or heterocycloalkyl moiety may optionally be substituted on the aryl, heteroaryl, cycloalkyl or heterocycloalkyl moiety with 1 to about 5 substituents independently selected from a halo, a cyano, nitro or hydroxyl group, a C₁ to C₆ alkyl group, or a C₁ to C₆ alkoxy group.

In one embodiment of the present invention, R₁ of formula (I) is preferably hydrogen or a C₁ to C₄ alkyl group and more preferably H.

In another embodiment of the present invention A of formula (I) is preferably an alkylenyl group, —(CH₂)ₙ—, where n is 1 to 3, more preferably 1 to 2 and most preferably 2.

In another embodiment, when it is desired to form a derivative of [2-(8,9-dioxo-2,6-diazabicyclo[5.2.0]non-1(7)-en-2-yl)alkyl]phosphonic acid, preferably at least one of R₂ and R₃ is not H.

In other embodiments, R₂ and R₃ of formula (I) are H or the moiety (B) or (D), more preferably, H or the moiety (B), and most preferably both are the moiety (B), where R₄, R₅ and R₆ are defined as above. When both R₂ and R₃ are not hydrogen, it is preferred that they be the same.

In another preferred embodiment of the present invention, both R₂ and R₃ are preferably hydrogen.

With respect to the moieties (B), (C), and (D), R₄ and R₅ are preferably H or a C₁ to C₄ alkyl group, and more preferably H or methyl. R₆ is preferably a C₃ to C₁₀ linear or branched alkyl group, a C₅ to C₇ aryl group, a 5- to 7-membered heteroaryl group, or a cycloalkyl group having in the ring 5 to 7 carbon atoms. In a preferred embodiment, R₆ is a C₅ to C₇ aryl group.

In yet another preferred embodiment of the present invention R₁ is H or a C₁ to C₄ alkyl group; A is an alkylenyl group having the formula —(CH₂)ₙ—, where n is 1 to 3; R₂ and R₃ are independently H or: 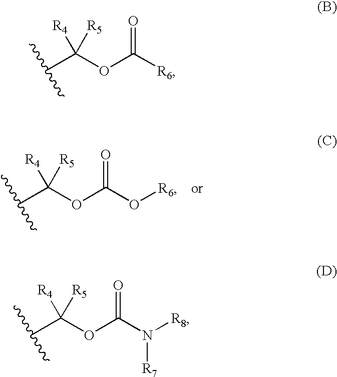
R₄ and R₅ are independently H or a C₁ to C₄ alkyl group; and R₆ is a C₃ to C₁₀ linear or branched alkyl group, a C₅ to C₇ aryl group, a 5- to 7-membered heteroaryl group, or a cycloalkyl group having in the ring 5 to 7 carbon atoms.

Specific examples of compounds useful in the present invention include the following compounds:
[2-(8,9-dioxo-2,6-diazabicyclo[5.2.0]non-1(7)-en-2-yl)ethyl]phosphonic acid;
3-{2-[8,9-dioxo-2,6-diazabicyclo[5.2.0]non-1(7)-en-2-yl]ethyl}-3-oxido-7-oxo-7-phenyl-2,4,6-trioxa-3-phosphahept-1-yl benzoate;
3-{2-[8,9-dioxo-2,6-diazabicyclo[5.2.0]non-1(7)-en-2-yl]ethyl}-3-oxido-7-oxo-8-propyl-2,4,6-trioxa-3-phosphaundec⁻¹-yl 2-propylpentanoate;
2,2-dimethyl-propionic acid (2,2-dimethyl-propionyloxymethoxy)-[2-(8,9-dioxo-2,6-diaza-bicyclo[5.2.0]non-1(7)-en-2-yl)-ethyl]-phosphinoyloxymethyl ester;
7-cyclohexyl-3-{2-[8,9-dioxo-2,6-diazabicyclo[5.2.0]non-1(7)-en-2-yl]ethyl}-1,5-dimethyl-3-oxido-7-oxo-2,4,6-trioxa-3-phosphahept-1-yl cyclohexanecarboxylate;
7-cyclohexyl-3-{2-[8,9-dioxo-2,6-diazabicyclo[5.2.0]non-1(7)-en-2-yl]ethyl}-3-oxido-7-oxo-2,4,6-trioxa-3-phosphahept-1-yl cyclohexanecarboxylate;
[2-(8,9-dioxo-2,6-diaza-bicyclo[5.2.0]non-1-(7)-en-2-yl)-ethyl]-phosphonic acid diisopropoxycarbonyl oxymethyl ester;
[2-[8,9-dioxo-2,6-diazabicyclo[5.2.0]non-1(7)-en-2-yl]ethyl]-phosphonic acid bis [1-(benzoyloxy)ethyl] ester;
benzoic acid [2-(8,9-dioxo-2,6-diaza-bicyclo[5.2.0]non-1(7)-en-2-yl)-ethyl]-hydroxy-phosphinoyloxymethyl ester; and
[2-(8,9-dioxo-2,6-diaza-bicyclo[5.2.0]non-1(7)-en-2-yl)-ethyl]-phosphonic acid di-dimethyl carbamoyloxymethyl ester;
   or a pharmaceutically acceptable salt thereof.

The compounds useful in this invention may contain asymmetric carbon atoms and/or phosphorus atoms, and thus can give rise to optical isomers and diastereoisomers. While shown without respect to stereochemistry in formula (I), the present invention includes such optical isomers and diastereoisomers; as well as the racemic and resolved, enantiomerically pure R and S stereoisomers; as well as other mixtures of the R and S stereoisomers and pharmaceutically acceptable salts thereof.

Where an enantiomer is preferred, it may, in some embodiments, be provided substantially free of the corresponding enantiomer. Thus, an enantiomer substantially free of the corresponding enantiomer refers to a compound that is isolated or separated via separation techniques or prepared free of the corresponding enantiomer. “Substantially free,” as used herein, means that the mixture of the various forms of the enantiomers is made up of a significantly greater proportion of one enantiomer. In preferred embodiments, the mixture comprises at least about 90% by weight of a preferred enantiomer. In other embodiments of the invention, the mixture comprises at least about 99% by weight of a preferred enantiomer. Preferred enantiomers may be isolated from racemic mixtures by any method known to those skilled in the art, including high performance liquid chromatography (HPLC) and the formation and crystallization of chiral salts or prepared by methods described herein. See, for example, Jacques, et al., Enantiomers, Racemates and Resolutions (Wiley Interscience, New York, 1981); Wilen, S. H., et al., Tetrahedron, 33:2725 (1977); Eliel, E. L. Stereochemistry of Carbon Compounds, (McGraw-Hill, NY, 1962); Wilen, S. H. Tables of Resolving Agents and Optical Resolutions, p. 268 (E. L. Eliel, Ed., Univ. of Notre Dame Press, Notre Dame, Ind. 1972).

One skilled in the art will also recognize that it is possible for tautomers to exist of formula (I). The present invention includes the use of all such tautomers even though not shown in formula (I).

The compounds useful in the present invention also include pharmaceutically acceptable salts of the compounds of formula (I). By “pharmaceutically acceptable salt,” it is meant any compound formed by the addition of a pharmaceutically acceptable base and a compound of formula (I) to form the corresponding salt. By the term “pharmaceutically acceptable,” it is meant a substance that is acceptable for use in pharmaceutical applications from a toxicological perspective and does not adversely interact with the active ingredient. Preferably, the pharmaceutically acceptable salts are alkali metal (sodium, potassium, lithium) or alkaline earth metal (calcium, magnesium) salts of the compounds of formula (I), or salts of the compounds of formula (I) with pharmaceutically acceptable cations derived from ammonia or a basic amine. Examples of the later include, but are not limited to, ammonium, mono-, di-, or trimethylammonium, mono-, di-, or triethylammonium, mono-, di, or tripropylammonium (iso and normal), ethyldimethylammonium, benzyldimethylammonium, cyclohexylammonium, benzylammonium, dibenzylammonium, piperidinium, morpholinium, pyrrolidinium, piperazinium, 1-methylpiperidinium, 1-isopropylpyrrolidinium, 1,4-dimethylpiperazinium, 1-n-butylpiperidinium, 2-methylpiperidinium, 1-ethyl-2-methylpiperidinium, mono-, di-, or triethanolammonium, tris-(hydroxymethyl)methylammonium, or phenylmonoethanolammonium. Preferably, salts may be formed when one of R₂ or R₃ is hydrogen.

The compounds useful in the present invention can be prepared by synthesizing the compound of the formula (II), where A and R₁ are defined as for formula (I): 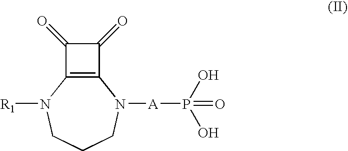
according to methods described in U.S. Pat. No. 5,168,103, U.S. Pat. No. 5,240,946, U.S. Pat. No. 5,990,307 and U.S. Pat. No. 6,011,168, the contents of which are entirely incorporated herein by reference. A preferred synthesis route is described in Example 5 of U.S. Pat. No. 5,990,307 and U.S. Pat. No. 6,011,168.

To form compounds where at least one of R₂ or R₃ is not hydrogen in formula (I), the compound of formula (II) obtained is then dissolved in a suitable solvent, such as dimethylformamide. As used herein, “suitable solvent” means that the compound of formula (II) is soluble therein and nonreactive therewith. Preferably, an acid scavenger (to react with the acid halide reaction by-product) such as an amine, is added to the reaction mixture at preferably ambient temperature. The amine is preferably a sterically hindered secondary or tertiary amine and more preferably a tertiary amine such as diisopropylethylamine. An appropriately substituted haloester of the formula: 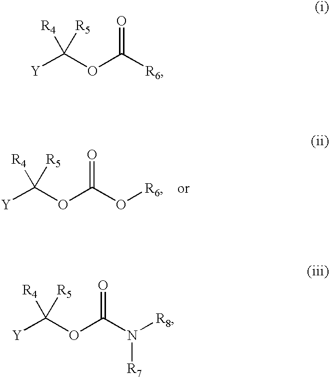
where R₄, R₅, and R₆ are defined as in formula (I), and Y is a halo atom, is added to the reaction mixture. The reaction mixture is heated from about 50° C. to about 80° C., and more preferably from about 65° C. to about 75° C. for a sufficient reaction time so that the halo ester reacts with the compound of formula (II) to form a compound of formula (I). Typically, for preferable yields, the reaction time is from about 20 hours to about 40 hours, and more preferably from about 25 hours to about 35 hours. After the reaction is complete, the reaction mixture is preferably cooled to ambient temperature, and the compound of formula (I) is isolated using standard techniques known to those skilled in the art. A preferred isolation method is to partition the reaction mixture between a mild base, such as aqueous sodium bicarbonate, and an organic solvent such as ethyl acetate. The aqueous phase is preferably several times re-extracted with the organic solvent, and the combined organic layers are washed again with a mild base. The organic layers are then dried, for example with brine and over magnesium sulfate, filtered and evaporated. The residue is then preferably flash chromatographed on silica gel using standard techniques to isolate the compound.

The compound of formula (I) is present in the solid, pharmaceutical dosage form in an effective amount for oral administration. As used herein, “an effective amount” is at least the minimal amount of the compound of formula (I) or a pharmaceutically acceptable salt form thereof, which treats the condition in question in a mammal. The effective amount will depend on such variables as the particular composition used, the severity of the symptoms, and the particular patient being treated. To determine the effective amount of the compound to be administered, the physician may, for example, evaluate the effects of a given compound of formula (I) in the patient by incrementally increasing the dosage until the desired symptomatic relief level is achieved. The continuing dose regimen may then be modified to achieve the desired result. For oral administration, preferably the compounds of the present invention are incrementally increased in a patient in an amount of from 1 mg/kg to 10 mg/kg until the desired symptomatic relief level is achieved. The continuing dose regimen may then be modified to achieve the desired result, with the range for oral dosage being preferably from about 200 mg/day to about 4000 mg/day, more preferably, about 400 mg/day to about 3200 mg/day, even more preferably at least about 800 mg/day, yet even more preferably at least about 1600 mg/day, and further even more preferably at least about 3200 mg/day. The patient may be administered the compounds of the present invention as a single oral dose (e.g., one 600 mg tablet or capsule) or as a multiple oral dose (e.g., three 200 mg tablets or capsules; two 300 mg tablets or capsules), preferably in the form of tablets or capsules.

In preferred embodiments, the compound of formula (I) is present in the solid, pharmaceutical dosage form at a level of about 25% by weight to about 99.5% by weight based on the total weight of said pharmaceutical composition, more preferably, at a level of about 50% by weight to about 99.5% by weight, based on the total weight of said pharmaceutical composition, even more preferably, at a level of about 60% by weight to about 99.5% by weight, based on the total weight of said solid, pharmaceutical dosage form, yet even more preferably, at a level of about 67% by weight to about 99.5% by weight, based on the total weight of said solid, pharmaceutical dosage form.

The solid, pharmaceutical dosage forms of the present invention, in addition to containing an effective amount of at least one compound of formula (I), preferably comprise at least one pharmaceutically acceptable absorption enhancer selected from the group consisting of: surfactant, bile salt, fatty acid, fatty acid salt, chelating agent, acyl carnitine, acyl choline, or a mixture thereof. In preferred embodiments, the absorption enhancer is present in the solid, pharmaceutical dosage form in an amount of from about 0.25% by weight to about 50% by weight, based on the total weight of said solid, pharmaceutical dosage form.

Suitable surfactants include, for example, ionic surfactant, nonionic surfactant or a mixture thereof. Exemplary ionic surfactants include sodium lauryl sulfate, dioctyl sodium sulfosuccinate or a mixture thereof. Exemplary nonionic surfactants include polyoxyethylene alkyl ether, polyoxyethylene alkyl ester, polysorbate or a mixture thereof.

Suitable polyoxyethylene alkyl esters include, for example, polyethylene glycol-20 sorbitan monooleate sold under the trade name TWEEN 80.

Suitable bile salts include, for example, sodium cholate, sodium deoxycholate, or a mixture thereof.

Suitable fatty acids include, for example, oleic acid. Suitable fatty acid salts include, for example, sodium caprate.

Suitable chelating agents include, for example, ethylenediaminetetraacetic acid (EDTA) and its salts, including sodium salts thereof.

Suitable acyl carnitines include, for example, palmitoyl carnitine. Suitable acyl cholines include, for example, lauroyl choline.

The solid, pharmaceutical dosage forms of the invention may optionally comprise at least one additive for forming a solid dosage form of said pharmaceutical composition. Suitable optional additives include fillers, disintegrants, binders, lubricants, or a mixture thereof. The absorbance enhancer may also serve the function of the sole additive or one of the additives for forming a solid dosage form.

Exemplary fillers include, for example, lactose, microcrystalline cellulose, mannitol, calcium phosphate, pregelatinized starch, pregelatinized sucrose, or a mixture thereof. Microcrystalline cellulose is preferred, especially as intragranulation and extragranulation component.

Exemplary disintegrants include, for example, croscarmellose sodium, starch, sodium starch glycolate, pregelatinized starch, crospovidone, and mixtures thereof. Croscarmellose sodium is preferred, especially as intragranulation and extragranulation component.

Exemplary binders include, for example, povidone (also known as polyvinyl pyrrolidone or PVP), hydroxypropylmethylcellulose, polyvinyl alcohol, gelatin, gum and mixtures thereof. Povidone is preferred. Preferably, binders if present, are included in the composition in an amount of preferably about 0.5% by weight to about 10% by weight, more preferably at least about 1.5% by weight, and most preferably at least about 2.5% by weight, based on the total weight of the composition.

Exemplary lubricants include, for example, magnesium stearate, sodium stearyl fumarate, and mixtures thereof.

Preferably, these additives for forming a solid dosage form in total will constitute at least about 0.25% by weight, more preferably from about 0.25% by weight to about 95% by weight, and most preferably from about 0.25% by weight to about 33% by weight, based on the total weight of the composition.

The solid, pharmaceutical dosage form may be prepared by conventional manufacturing techniques for forming oral solid dosage forms, including but not limited to wet, dry, fluid-bed granulation and direct compression techniques. Such techniques are described in Remington: The Science and Practice of Pharmacy, 20^{th} Edition (Easton, Pa.: Mack Publishing Company, 2000), pages 858-893, the disclosure of which is incorporated herein by reference in its entirety. The wet granulation technique employed in Examples 1 and 2 improved the density of the powder blend from 0.33 g/ml to 0.59 g/ml, permitting the encapsulation of 300 mg of active ingredient ([2-(8,9-dioxo-2,6-diazabicyclo[5.2.0]non-1(7)-en-2-yl)ethyl]phosphonic acid) in size #0 HPMC capsules.

The solid, pharmaceutical dosage form of the present invention may also optionally contain one or more antimicrobial preservatives to prevent microbial growth during storage and multiple dose use. Examples of suitable preservatives are benzalkonium chloride, thimersal, chlorobutanol, or parabens, or combinations thereof. Although the concentration of the preservative in the composition will depend upon the preservative used, preferably the total amount of preservative present in the composition will range from about 0.1% by weight to about 2.0%, by weight, based on the total weight of the composition.

In another embodiment of the present invention, the solid, pharmaceutical dosage form may contain one or more other pharmaceutical active agents such as those agents being used to treat any other medical condition present in the mammal. Examples of such pharmaceutical active agents include pain relieving agents, anti-angiogenic agents, anti-neoplastic agents, anti-diabetic agents, anti-infective agents, or gastrointestinal agents, or combinations thereof. A more complete listing of pharmaceutical active agent can be found in the Physicians' Desk Reference, 55th Edition, 2001, published by Medical Economics Co., Inc., Montvale, N.J. Each of these agents may be administered according to the therapeutically effective dosages and regimens known in the art, such as those described for the products in the Physicians' Desk Reference, 55th Edition, 2001, published by Medical Economics Co., Inc., Montvale, N.J.

This invention also includes kits or packages of pharmaceutical formulations designed for use in the regimens and methods described herein. These kits are preferably designed for daily oral administration over the specified term or cycle of administration, preferably for the number of prescribed oral administrations per day, and organized so as to indicate a single oral formulation or combination of oral formulations to be taken on each day of the regimen or cycle. Preferably, each kit will include oral tablets to be taken on each the days specified, in some embodiments one oral tablet will contain each of the combined daily dosages indicated and in other embodiments, the administrations of the separate compounds will be present in separate formulations or compositions. It is most preferable that the package or kit shall have a calendar or days-of-the-week designation directing the administration of the appropriate compositions on the appropriate day or time.

In another embodiment of the present invention, the present invention provides methods for treating one or more conditions associated with a glutamate abnormality that includes administering orally to a mammal in need thereof a therapeutically effective amount of at least one compound of formula (I). As used herein, “associated with” refers to conditions directly or indirectly caused by a glutamate abnormality. “Glutamate abnormality” refers to any condition produced by a disease or a disorder in which glutamate and/or its receptors are implicated as a contributing factor to the disease or disorder. Conditions believed to be associated with a glutamate abnormality include, but are not limited to, vascular disorders associated with a glutamate abnormality, such as cerebral vascular disorders including, but not limited to, cerebral ischemia (e.g., stroke) or cerebral infarction resulting in a range of conditions such as thromboembolic or hemorrhagic stroke, or cerebral vasospasm; cerebral trauma; muscular spasm; convulsive disorders such as epilepsy or status epilepticus; glaucoma; pain; anxiety disorders such as such as panic attack, agoraphobia, panic disorder, specific phobia, social phobia, obsessive compulsive disorder, posttraumatic stress disorder, acute stress disorder, generalized anxiety disorder, separation anxiety disorder, or substance-induced anxiety disorder; mood disorders such as bipolar disorders (e.g., bipolar I disorder, bipolar II disorder, and cyclothymic disorder), depressive disorders (e.g., major depressive disorder, dysthymic disorder, or substance-induced mood disorder), mood episodes (e.g., major depressive episode, manic episode, mixed episode, and hypomanic episode); schizophrenia; schizophreniform disorder; schizoaffective disorder; cognitive impairment such as memory loss; and chronic neurodegenerative disorders such as Parkinson's disease, Huntington's disease, Alzheimer's disease, amyotrophic lateral sclerosis, or chronic dementia related to, for example, Lewy body disease, Alzheimer's disease, fronto temporal dementia, or AIDS. With respect to the mental disorders listed above such as schizophrenia, mood disorders and anxiety disorders, reference is made to the Diagnostic and Statistical Manual of Mental Disorders, 4^{th} edition, Washington, D.C., American Psychiatric Association (1994) for a more complete description of each of the mental disorder. Additional conditions believed to be related to glutamate abnormalities include inflammatory diseases; hypoglycemia; diabetic end organ complications; cardiac arrest; asphyxia anoxia, such as from near drowning, pulmonary surgery and cerebral trauma; and spinal cord injury. The compounds of the present invention may also be used to treat fibromyalgia, irritable bowel syndrome, and complications from herpes zoster (shingles) such as prevention of post-herpetic neuralgia. The pharmaceutical compositions in the present invention may also be used to prevent tolerance to opiate analgesia or to help control symptoms of withdrawal from addictive drugs. Thus, the present invention provides methods for treating each of the aforementioned conditions that includes administering orally to a mammal in need thereof a therapeutically effective amount of at least one compound of formula (I).

In one preferred embodiment, the compounds useful in the present invention are used to treat pain. The pain may be, for example, acute pain (short duration) or chronic pain (reoccurring or persistent). The pain may also be centralized or peripheral.

Examples of pain that can be acute or chronic and that can be treated in accordance with the methods of the present invention include inflammatory pain, musculoskeletal pain, bony pain, lumbosacral pain, neck or upper back pain, visceral pain, somatic pain, neuropathic pain, cancer pain, pain caused by injury or surgery such as burn pain or dental pain, or headaches such as migraines or tension headaches, or combinations of these pains. One skilled in the art will recognize that these pains may overlap one another. For example, a pain caused by inflammation may also be visceral or musculoskeletal in nature.

In a preferred embodiment of the present invention the compounds useful in the present invention are administered in mammals to treat chronic pain such as neuropathic pain associated for example with damage to or pathological changes in the peripheral or central nervous systems; cancer pain; visceral pain associated with for example the abdominal, pelvic, and/or perineal regions or pancreatitis; musculoskeletal pain associated with for example the lower or upper back, spine, fibromylagia, temporomandibular joint, or myofascial pain syndrome; bony pain associated with for example bone or joint degenerating disorders such as osteoarthritis, rheumatoid arthritis, or spinal stenosis; headaches such migraine or tension headaches; or pain associated with infections such as HIV, sickle cell anemia, autoimmune disorders, multiple sclerosis, or inflammation such as osteoarthritis or rheumatoid arthritis.

In a more preferred embodiment, the compounds useful in this invention are used to treat chronic pain that is neuropathic pain, visceral pain, musculoskeletal pain, bony pain, cancer pain or inflammatory pain or combinations thereof, in accordance with the methods described herein. Inflammatory pain can be associated with a variety of medical conditions such as osteoarthritis, rheumatoid arthritis, surgery, or injury. Neurophathic pain includes peripheral neuropathic pain, central neuropathic pain or combinations thereof. Neuropathic pain may be associated with for example diabetic neuropathy, post-herpetic neuralgia, trigeminal neuralgia, complex regional pain syndrome, lumbar or cervical radiculopathies, fibromyalgia, glossopharyngeal neuralgia, reflex sympathetic dystrophy, causalgia, thalamic syndrome, nerve root avulsion, monoclonal gammopathy of undetermined significance (MGUS) neuropathy, sarcoid polyneuropathy, HIV-related neuropathy arising from a variety of causes such as from medication used to treat HIV, peripheral neuropathy such as peripheral neuropathy with connective tissue disease, paraneoplastic sensory neuropathy, familial amyloid polyneuropathy, acquired amyloid polyneuropathy, inherited neuropathy, neuropathy with renal failure, hereditary sensory autonomic neuropathy, Fabry's disease, Celiac disease or nerve damage cause by injury resulting in peripheral and/or central sensitization such as phantom limb pain, reflex sympathetic dystrophy or postthoracotomy pain, cancer including neuropathies caused by chemotherapy agents or other agents used to treat the disease, chemical injury, toxins such as arsenic neuropathy, nutritional deficiencies, or viral or bacterial infections such as shingles or HIV-related neuropathy, or combinations thereof. The methods of use for compounds of this invention further include treatments in which the neuropathic pain is a condition secondary to metastatic infiltration, adiposis dolorosa, burns, or central pain conditions related to thalamic conditions.

Neuropathic pains described above may also be, in some circumstances, classified as “painful small fiber neuropathies” such as idiopathic small-fiber painful sensory neuropathy, or “painful large fiber neuropathies” such as demylinating neuropathy or axonal neuropathy, or combinations thereof. Such neuropathies are described in more detail, for example, in the J. Mendell et al., N. Engl. J. Med. 2003, 348:1243-1255, which is hereby incorporated by reference in its entirety.

As mentioned previously, the methods of the present invention may be used to treat pain that is somatic and/or visceral in nature. For example, somatic pain that can be treated in accordance with the methods of the present invention include pains associated with structural or soft tissue injury experienced during surgery, dental procedures, burns, or traumatic body injuries. Examples of visceral pain that can be treated in accordance with the methods of the present invention include those types of pain associated with or resulting from maladies of the internal organs such as ulcerative colitis, irritable bowel syndrome, irritable bladder, Crohn's disease, rheumatologic (arthralgias), tumors, gastritis, pancreatitis, infections of the organs, or biliary tract disorders, or combinations thereof. One skilled in the art will also recognize that the pain treated according to the methods of the present invention may also be related to conditions of hyperalgesia, allodynia, or both. Additionally, the chronic pain may be with or without peripheral or central sensitization.

The compounds useful in this invention may also be used to treat acute and/or chronic pains associated with female conditions, which may also be referred to as female-specific pain. Such groups of pain include those that are encountered solely or predominately by females, including pain associated with menstruation, ovulation, pregnancy or childbirth, miscarriage, ectopic pregnancy, retrograde menstruation, rupture of a follicular or corpus luteum cyst, irritation of the pelvic viscera, uterine fibroids, adenomyosis, endometriosis, infection and inflammation, pelvic organ ischemia, obstruction, intra-abdominal adhesions, anatomic distortion of the pelvic viscera, ovarian abscess, loss of pelvic support, tumors, pelvic congestion or referred pain from non-gynecological causes.

The term “treat” or “treating”, as used herein, in addition to partially or completely alleviating pain that has already developed in a mammal, is also meant to include totally or partially inhibiting (i.e., preventing) the development of pain. Thus, compounds of the present invention may be administered to a mammal prior to the mammal experiencing pain to partially or totally inhibit the development of pain.

In one embodiment, the compounds useful in the present invention may be administered prior to or during a surgical procedure to partially or totally inhibit development of pain associated with the surgical procedure. In a preferred embodiment, the compounds useful in the present invention are preferably administered from about 0.25 hours to about 4 hours prior to the surgical procedure. For surgical procedures of greater duration, dosing is preferably repeated during the surgical procedure about every time interval corresponding to the in vivo half life (T_{1/2}) of the compound. In a preferred embodiment, for formulations according to example 1, dosing is repeated about every 4 to 8 hours during the surgical procedure.

In another embodiment of the present invention, it has been found that administering compounds useful in the present invention prior to a surgical procedure may increase the potency and/or effectiveness of other pain relieving agents such as opioid analgesics (e.g., morphine) that are administered after the surgical procedure, and/or may reduce the amount of pain relieving agent needed to treat the post operative surgical pain. Thus, the present invention provides methods of treating pain associated with a surgical procedure that includes administering prior to or during the surgical procedure a compound useful in the present invention, and administering after or during a surgical procedure a therapeutically effective amount of at least one pain relieving agent, such as an opioid analgesic. In preferred embodiments, compounds of the present invention may be administered to a mammal also after the surgical procedure, preferably in combination with the one or more pain relieving agents. “Surgical procedure,” as used herein include all therapeutic, diagnostic, and/or cosmetic manipulations, disruptions, movements, radiations, ablations, chemical or physical alterations in any tissue, organ, or body system including but not limited to blood, blood vessels, fat, skin, connective tissue, muscle, internal organs, glands, bone, cartilage, nerve, marrow, fascia, meninges, sensory apparatus, brain or spinal cord. Surgical procedure includes, for example, procedures performed on mammals using more recent surgical techniques such as laser, ultrasound, and radiation in addition to more traditional techniques.

In another embodiment, the compounds useful in the present invention may be administered to totally or partially inhibit a neuropathic pain condition from developing. For example, compounds of the present invention may be administered to a mammal who is at risk for developing a neuropathic pain condition such as a mammal who has contracted shingles or a mammal who is being treated for cancer.

In another embodiment of the present invention, the compounds useful in the present invention may be administered to a mammal with one or more other pharmaceutical active agents such as those agents being used to treat any other medical condition present in the mammal. Examples of such pharmaceutical active agents include pain relieving agents, anti-angiogenic agents, anti-neoplastic agents, anti-diabetic agents, anti-infective agents, or gastrointestinal agents, or combinations thereof.

The one or more other pharmaceutical active agents may be administered in a therapeutically effective amount simultaneously (such as individually at the same time, or together in a pharmaceutical composition), and/or successively with one or more compounds of the present invention.

The method of administration of the other pharmaceutical active agent may be the same or different from the route of administration used for the compounds of the present invention. For example, the other pharmaceutical active agents may be administered by oral or parental administration, such as for example, by intramuscular, intraperitoneal, epidural, intrathecal, intravenous, intramucosal, such as by intranasal or sublingual, subcutaneous or transdermal administration. The preferred administration route will depend upon the particular pharmaceutical active agent chosen and its recommended administration route(s) known to those skilled in the art.

A more complete listing of pharmaceutical active agent can be found in the Physicians' Desk Reference, 55th Edition, 2001, published by Medical Economics Co., Inc., Montvale, N.J. Each of these agents may be administered according to the therapeutically effective dosages and regimens known in the art, such as those described for the products in the Physicians' Desk Reference, 55th Edition, 2001, published by Medical Economics Co., Inc., Montvale, N.J.

In a preferred embodiment of the present invention, the compounds useful in the present invention may be administered to a mammal with one or more other pain relieving agents to treat pain in a mammal. By “pain relieving agents,” it is meant any agent that directly or indirectly treats pain symptoms. Examples of indirect pain relieving agents include for example anti-inflammatory agents, such as anti-rheumatoid agents.

The one or more other pain relieving agents may be administered simultaneously (such as individually at the same time, or together in a pharmaceutical composition), and/or successively with the compounds of the present invention. Preferably, the compounds of the present invention and the one or more pain relieving agents are administered in a manner so that both are present in the mammal body for a certain period of time to treat pain.

The method of administration of the other pain relieving agent may be the same or different from the route of administration used for the compound of the present invention. For example, opioids are preferably administered by oral, intravenous, intranasal, or intramuscular administration routes.

One skilled in the art will recognize that the dosage of the other pain relieving agent administered to the mammal will depend on the particular pain relieving agent in question and the desired administration route. Accordingly, the other pain relieving agent may be dosed and administered according to those practices known to those skilled in the art such as those disclosed in references such as the Physicians' Desk Reference, 55th Edition, 2001, published by Medical Economics Co., Inc., Montvale, N.J.

Examples of pain relieving agents that may be administered with the compound of the present invention include analgesics such as non-narcotic analgesics or narcotic analgesics; anti-inflammatory agents such as non-steroidal anti-inflammatory agents (NSAID), steroids or anti-rheumatic agents; migraine preparations such as beta adrenergic blocking agents, ergot derivatives, or isometheptene; tricyclic antidepressants such as amitryptyline, desipramine, or imipramine; anti-epileptics such as gabapentin, carbamazepine, topiramate, sodium valproate or phenyloin; α₂ agonists; or selective serotonin reuptake inhibitors/selective norepinepherine uptake inhibitors, or combinations thereof. One skilled in the art will recognize that some agents described hereinafter act to relieve multiple conditions such as pain and inflammation, while other agents may just relieve one symptom such as pain. A specific example of an agent having multiple properties is aspirin, where aspirin is anti-inflammatory when given in high doses, but at lower doses is just an analgesic. The pain-relieving agent may include any combination of the aforementioned agents, for example, the pain-relieving agent may be a non-narcotic analgesic in combination with a narcotic analgesic.

In a preferred embodiment of the present invention, at least one compound of the present invention is administered with at least one opioid analgesic in accordance with the methods previously described herein to treat pain. It has been found that the compounds of the present invention, when administered with at least one opioid analgesic such as morphine, have such beneficial effects as decreasing pain perception, increasing the duration of pain relief, and/or decreasing adverse side effects to a greater extent than other comparator NMDA antagonists.

The present invention will now be illustrated by reference to the following specific, non-limiting examples. Those skilled in the art of organic synthesis may be aware of still other synthetic routes to the invention compounds. The reagents and intermediates used herein are either commercially available or prepared according to standard literature procedures.

In another embodiment, the invention is directed to processes for forming a formulation containing the compounds of formula (I). The processes include the steps of forming a wet granulation; and forming a solid dosage form. The wet granulation contains:
at least one binder, preferably povidone;
optionally at least one filler, preferably microcrystalline cellulose;
optionally at least one disintegrant, preferably croscarmellose sodium; and
at least one compound of formula (I) or a pharmaceutically acceptable salt thereof, preferably, [2-(8,9-dioxo-2,6-diazabicyclo[5.2.0]non-1(7)-en-2-yl)ethyl]phosphonic acid or a pharmaceutically acceptable salt thereof.

In certain preferred embodiments, the wet granulation is formed by dry blending at least one filler or disintegrant with said compound of formula (I) or a pharmaceutically acceptable salt thereof; and then granulating the dry blend with a solution of at least one binder to form a wet granulation.

In certain preferred embodiments, the processes further include the steps of: drying the wet granulation; milling the dried granulation; and then optionally blending said milled, dried granulation with one or more extragranulation components, preferably including the filler and/or disintegrant added to form the wet granulation.

In certain embodiments, the invention is directed to the product produced by the above-described processes.

The present invention will now be illustrated by reference to the following specific, non-limiting examples. Those skilled in the art of organic synthesis may be aware of still other synthetic routes to the invention compounds. The reagents and intermediates used herein are either commercially available or prepared according to standard literature procedures.

### EXAMPLES

The present invention is further defined in the following Examples, in which all parts and percentages are by weight and degrees are Celsius, unless otherwise stated. It should be understood that these examples, while indicating preferred embodiments of the invention, are given by way of illustration only. From the above discussion and these examples, one skilled in the art can ascertain the essential characteristics of this invention, and without departing from the spirit and scope thereof, can make various changes and modifications of the invention to adapt it to various usages and conditions.

### Example 1

### Capsule Formulations (69.4% [2-(8,9-dioxo-2,6-diazabicyclo[5.2.0]non-1(7)-en-2-yl)ethyl]phosphonic Acid)

Three strengths (100, 200, 300 mg capsules) were manufactured from one common wet granulation. The batch size of granulation was 1297.8 g. The formulae of all of the strengths of [2-(8,9-dioxo-2,6-diazabicyclo[5.2.0]non-1(7)-en-2-yl)ethyl]phosphonic acid capsules are shown in Table 1.

A mixture of the intragranular part of microcrystalline cellulose, [2-(8,9-dioxo-2,6-diazabicyclo[5.2.0]non-1(7)-en-2-yl)ethyl]phosphonic acid and croscarmellose sodium was prepared. A solution of povidone in purified water was prepared by dissolving the povidone is purified water. The mixture was granulated with the povidone solution in a high shear granulator. Additional purified water was added, as needed, to achieve desired granulation end point. The granulation was then dried in a suitable dryer, milled, and transferred into a blender. Microcrystalline cellulose and croscarmellose sodium were added to the granulation and blended. Magnesium stearate was added and blended. A capsule-filling machine was set up with parts for filling #0 capsules. The [2-(8,9-dioxo-2,6-diazabicyclo[5.2.0]non-1(7)-en-2-yl)ethyl]phosphonic acid (69.35% by weight, based on the total weight of the formulation) with brown opaque hydroxypropylmethyl cellulose (HPMC) capsule #0 using the target fill weight.

The analytical data for these capsules are shown in Table 2.

TABLE 1

| | 100 mg | 200 mg | 300 mg |
|---|---|---|---|
| Ingredient | mg/cap | mg/cap | mg/cap |
| Intragranular | | | |
| [2-(8,9-dioxo-2,6- | 100.00 | 200.00 | 300.00 |
| diazabicyclo[5.2.0]non-1(7)-en- | | | |
| 2-yl)ethyl]phosphonic acid | | | |
| Microcrystalline cellulose | 13.91 | 27.82 | 41.73 |
| (AVICEL PH 101) | | | |
| Povidone USP, 17 PF | 3.61 | 7.22 | 10.83 |
| Croscarmellose sodium | 5.77 | 11.54 | 17.31 |
| Extragranular | | | |
| Microcrystalline cellulose | 14.42 | 28.84 | 43.26 |
| (AVICEL PH 101) | | | |
| Croscarmellose sodium | 5.77 | 11.54 | 17.31 |
| Magnesium stearate (vegetable | 0.72 | 1.44 | 2.16 |
| grade) | | | |
| Total | 144.20 | 288.40 | 432.60 |

TABLE 2

| Test | | Result | | Result | | Result | |
|---|---|---|---|---|---|---|---|
| | | 100 mg | | 200 mg | | 300 mg | |
| Strength (HPLC) | | 98.3% LC | | 97.5% LC | | 98.9% LC | |
| Content Uniformity | | Ave: 96.8% | | Ave: 98.0% | | Ave: 99.7% | |
| | | cv = 3.8% | | cv = 2.7% | | cv = 1.5% | |
| | | Range: | | Range: | | Range: | |
| | | 89.9-101.2% | | 94.5-101.8% | | 98.1-102.6% | |
| | | Avg. | Range | Avg. | Range | Avg. | Range |
| Dissolution | Time | % | % | % | % | % | % |
| | 15 | 97.0 | 95-99 | 98.6 | 97-102 | 96.2 | 89-100 |
| | minutes | | | | | | |
| | 30 | 98.8 | 96-102 | 99.7 | 98-102 | 99.1 | 93-102 |
| | minutes | | | | | | |
| | 45 | 99.5 | 96-102 | 99.8 | 98-102 | 99.6 | 95-103 |
| | minutes | | | | | | |

LC = Label claim
RL = Method reporting limit

### Example 2

### Capsule Formulations (86.7% [2-(8,9-dioxo-2,6-diazabicyclo[5.2.0]non-1(7)-en-2-yl)ethyl]phosphonic Acid)

The manufacturing process of Example 1 was repeated using the following ingredients:

| | 200 mg |
|---|---|
| Ingredient | mg/capsule |
| Intragranular | |
| [2-(8,9-dioxo-2,6- | 200.00 |
| diazabicyclo[5.2.0]non-1(7)-en-2- | |
| yl)ethyl]phosphonic acid | |
| Povidone USP, 17 PF | 3.53 |
| Croscarmellose sodium | 7.05 |
| Microcrystalline cellulose | 14.1 |
| (AVICEL PH 101) | |
| Extragranular | |
| Croscarmellose sodium | 4.7 |
| Magnesium stearate (vegetable grade) | 1.18 |
| Total | 230.56 |

### Example 3

### Capsule Formulations (69.35% [2-(8,9-dioxo-2,6-diazabicyclo[5.2.0]non-1(7)-en-2-yl)ethyl]phosphonic Acid)

The manufacturing process of Example 1 was repeated using the following ingredients:

| | 300 mg |
|---|---|
| Ingredient | mg/capsule |
| Intragranular | |
| [2-(8,9-dioxo-2,6- | 208.05 |
| diazabicyclo[5.2.0]non-1(7)-en-2- | |
| yl)ethyl]phosphonic acid | |
| Povidone USP, 17 PF | 7.5 |
| Croscarmellose sodium | 12.00 |
| Microcrystalline cellulose | 28.95 |
| (AVICEL PH 101) | |
| Extragranular | |
| Microcrystalline cellulose | 30.00 |
| (AVICEL PH 101) | |
| Croscarmellose sodium | 12.00 |
| Magnesium stearate (vegetable grade) | 1.5 |
| Total | 300 |

A common granulation containing 69.35% active ingredient was developed by wet granulation method. Capsules of 100 mg or 300 mg strengths were manufactured by filling 144.20 mg and 432.6 mg, respectively of the final blend in #0 capsules.

### Example 4

### Enteric-Coated Tablet Formulation

The formulation studies for tablets were carried out by a wet granulation method. A 200 mg of [2-(8,9-dioxo-2,6-diazabicyclo[5.2.0]non-1(7)-en-2-yl)ethyl]phosphonic acid tablet was prepared using povidone K17 (USP) as binder and croscarmellose sodium (Ac-Di-Sol™ available from FMC Corporation) as disintegrant. The tablets were then coated by enteric coat solution. This tablet was used as base formulation of [2-(8,9-dioxo-2,6-diazabicyclo[5.2.0]non-1(7)-en-2-yl)ethyl]phosphonic acid 200 mg tablet.

| | Input/Tablet | |
|---|---|---|
| Ingredients | (mg) | Function |
| Intragranular | | |
| 2-(8,9-dioxo-2,6- | 200.00 | Active ingredient |
| diazabicyclo[5.2.0] | | |
| non-1(7)-en-2-yl)ethyl] | | |
| phosphonic acid | | |
| Croscarmellose sodium | 7.05 | Disintegrant |
| Povidone USP, 17 PF | 3.53 | Binder |
| Extragranular | | |
| Microcrystalline cellulose | 14.10 | Diluent and disintegrant |
| (AVICEL PH 101) | | |
| Croscarmellose sodium | 4.70 | Disintegrant |
| Magnesium stearate | 1.18 | Lubricant |

The tablets remained intact for 2 hours in 0.01N HCl for 2 hours. The tablets disintegrated completely within 26 minutes in phosphate buffer (pH 6.8).

### Example 5

### Enteric-Coated Tablet Formulation Containing Sodium Lauryl Sulfate

An enteric-coated tablet formulation containing sodium lauryl sulfate was prepared in accordance with following table:

| | Input/Tablet | |
|---|---|---|
| Ingredients | (mg) | Function |
| Intragranular | | |
| 2-(8,9-dioxo-2,6- | 200.00 | Active ingredient |
| diazabicyclo[5.2.0] | | |
| non-1(7)-en-2-yl)ethyl] | | |
| phosphonic acid | | |
| Croscarmellose sodium | 7.05 | Disintegrant |
| Povidone USP, 17 PF | 3.53 | Binder |
| Extragranular | | |
| Microcrystalline cellulose | 14.10 | Diluent and disintegrant |
| (AVICEL PH 101) | | |
| Croscarmellose sodium | 4.70 | Disintegrant |
| Sodium lauryl sulfate | 5.88 | Absorption enhancer |
| Magnesium stearate | 1.18 | Lubricant |

Visulution of this tablet was performed in 0.01N HCl for 2 hours and then in phosphate buffer (pH 6.8) until tablet disintegrated completely and visulution time in buffer was 24 minutes.

### Example 6

### Enteric Coated Tablet Formulation Containing EDTA Tetra Sodium

An enteric-coated tablet formulation containing EDTA tetra sodium was prepared in accordance with following table:

| | Input/Tablet | |
|---|---|---|
| Ingredients | (mg) | Function |
| Intragranular | | |
| 2-(8,9-dioxo-2,6- | 200.00 | Active ingredient |
| diazabicyclo[5.2.0] | | |
| non-1(7)-en-2-yl)ethyl] | | |
| phosphonic acid | | |
| Croscarmellose sodium | 7.05 | Disintegrant |
| Povidone USP, 17 PF | 3.53 | Binder |
| Extragranular | | |
| Microcrystalline cellulose | 14.10 | Diluent and disintegrant |
| (AVICEL PH 101) | | |
| Croscarmellose sodium | 4.70 | Disintegrant |
| EDTA tetra sodium | 7.05 | Absorption enhancer |
| Magnesium stearate | 1.18 | Lubricant |

Visulution of this tablet was performed in 0.01N HCl for 2 hours and then in Phosphate buffer (pH 6.8) until tablet disintegrate completely and visulution time in buffer was 26 minutes.

### Example 7

### Enteric Coated Tablet Formulation Containing TWEEN 80

An enteric-coated tablet formulation containing TWEEN 80 was prepared in accordance with following table:

| | Input/Tablet | |
|---|---|---|
| Ingredients | (mg) | Function |
| Intragranular | | |
| 2-(8,9-dioxo-2,6- | 200.00 | Active ingredient |
| diazabicyclo[5.2.0] | | |
| non-1(7)-en-2-yl)ethyl] | | |
| phosphonic acid | | |
| Microcrystalline cellulose | 29.00 | Diluent and disintegrant |
| (AVICEL PH 101) | | |
| Croscarmellose sodium | 9.00 | Disintegrant |
| Polyethylene glycol-20 | 15.00 | Absorption enhancer |
| sorbitan monooleate | | |
| (TWEEN-80) | | |
| Povidone USP, 17 PF | 10.50 | Binder |
| Extragranular | | |
| Microcrystalline cellulose | 29.00 | Diluent and disintegrant |
| (AVICEL PH 101) | | |
| Croscarmellose sodium | 6.00 | Disintegrant |
| Magnesium stearate | 1.50 | Lubricant |

Visulution of this tablet was performed in 0.01N HCl for 2 hours and then in Phosphate buffer (pH 6.8) until tablet disintegrate completely and visulution time in buffer was 15 minutes.

### Example 8

### Enteric Coated Tablet Formulation Containing Sodium Caprate

An enteric-coated tablet formulation containing sodium lauryl caprate was prepared in accordance with following table:

| | Input/Tablet | |
|---|---|---|
| Ingredients | (mg) | Function |
| Intragranular | | |
| 2-(8,9-dioxo-2,6- | 200.00 | Active ingredient |
| diazabicyclo[5.2.0] | | |
| non-1(7)-en-2-yl)ethyl] | | |
| phosphonic acid | | |
| Microcrystalline cellulose | 20.00 | Diluent and disintegrant |
| (AVICEL PH 101) | | |
| Croscarmellose sodium | 20.00 | Disintegrant |
| Sodium caprate | 50.00 | Absorption enhancer |
| Povidone USP, 17 PF | 18.00 | Binder |
| Extragranular | | |
| Microcrystalline cellulose | 82.00 | Diluent and disintegrant |
| (AVICEL PH 101) | | |
| Croscarmellose sodium | 8.00 | Disintegrant |
| Magnesium stearate | 2.00 | Lubricant |

### Example 9

### Capsule Formulation Containing Enteric Coated Tablet Formulation and Palmitoyl Carnitine

A capsule containing enteric coated tablet formulation and palmitoyl carnitine was prepared in accordance with following table:

| Ingredients | % (W/W) | mg/tablet |
|---|---|---|
| Intragranular | | |
| [2-(8,9-dioxo-2,6- | 86.75 | 200.00 |
| diazabicyclo[5.2.0]non-1(7)-en-2- | | |
| yl)ethyl]phosphonic acid | | |
| Croscarmellose sodium | 3.06 | 7.05 |
| Povidone USP, 17 PF | 1.53 | 3.53 |
| Extragranular | | |
| Croscarmellose sodium | 2.04 | 4.70 |
| Microcrystalline cellulose | 6.11 | 14.10 |
| (AVICEL PH 101) | | |
| Magnesium stearate | 0.51 | 1.18 |
| Core tablet weight | 100.00 | 230.56 |
| Enteric film coat weight | 8.00 | 18.44 |
| Final tablet weight | | 249.00 |
| Palmitoyl carnitine | | 200 |
| HGC#1 | | 1 Capsule (TIC) |

### Example 10

### Bioavailability of [2-(8,9-dioxo-2,6-diazabicyclo[5.2.0]non-1(7)-en-2-yl)ethyl]phosphonic Acid in Beagle Dogs: Evaluation of Oral Dosage Formulations

This study was undertaken to investigate the bioavailability of [2-(8,9-dioxo-2,6-diazabicyclo[5.2.0]non-1(7)-en-2-yl)ethyl]phosphonic acid in beagle dogs. Comparison of experimental formulations included an immediate release capsule formulation and seven enteric-coated formulations.

Twelve female beagle dogs were assigned to four groups (3 dogs/group). Studies included a two period crossover study. A one-week washout separated each treatment. Each group was administered a single 200 or 400 mg dose of [2-(8,9-dioxo-2,6-diazabicyclo[5.2.0]non-1(7)-en-2-yl)ethyl]phosphonic acid.

All formulations were administered with 10 ml water. Blood samples were drawn at times specified in the protocol via jugular venipuncture; plasma was separated, frozen and stored at −70° C. until analysis. The concentration of [2-(8,9-dioxo-2,6-diazabicyclo[5.2.0]non-1(7)-en-2-yl)ethyl]phosphonic acid in plasma was determined by a validated HPLC assay.

Noncompartmental analysis of the plasma [2-(8,9-dioxo-2,6-diazabicyclo[5.2.0]non-1(7)-en-2-yl)ethyl]phosphonic acid concentration-time profiles was performed. Cₘₐₓ and tₘₐₓ values were noted directly from the individual dog profiles and AUC (0-24) values were calculated using linear trapezoid rule. The results are shown in Table 3.

The results indicate that the enteric-coated formulations containing absorption enhancers provided greater [2-(8,9-dioxo-2,6-diazabicyclo[5.2.0]non-1(7)-en-2-yl)ethyl]phosphonic acid exposure compared to the immediate release capsules that do not contain absorption enhancers. The results also indicate that the enteric-coated formulations containing absorption enhancers provided greater [2-(8,9-dioxo-2,6-diazabicyclo[5.2.0]non-1(7)-en-2-yl)ethyl]phosphonic acid exposure compared to the enteric-coated capsules that do not contain absorption enhancers at equivalent doses. The mean dose-normalized ratios (AUC₀₋₂₄) of the enteric-coated formulations to immediate release capsules ranged from 1.20 to 2.51.

TABLE 3

| | Absorption | AUC₀₋₂₄ | AUC | Cₘₐₓ | Cₘₐₓ | tₘₐₓ | t_{lag} |
|---|---|---|---|---|---|---|---|
| Formulation | Enhancer | (μg · hr/mL) | Ratio^{a} | (μg/ml) | Ratio^{a} | (hour) | (hour) |
| 2 × 200 mg | None | 18.6 (6.17) | — | 6.20 | — | 1.33 | 0 |
| Immediate | | | | (4.53) | | (0.76) | |
| release capsule | | | | | | | |
| (Comparative) | | | | | | | |
| (Example 2) | | | | | | | |
| 1 × 200 mg | None | 5.24 (5.06) | 0.56 | 2.55 | 0.82 | 2.50 | 0.83 |
| Enteric-coated | | | | (2.34) | | (1.50) | (0.58) |
| tablets | | | | | | | |
| (Comparative) | | | | | | | |
| 2 × 200 mg | None | 22.3 (9.14) | 1.20 | 12.7 | 2.04 | 2.67 | 1.67 |
| Enteric-coated | | | | (6.89) | | (1.15) | (0.76) |
| tablets | | | | | | | |
| (Comparative) | | | | | | | |
| 2 × 200 mg | Polyethylene | 24.0 (14.0) | 1.29 | 14.5 | 2.34 | 1.67 | 0.67 |
| Enteric-coated | glycol-20 | | | (9.30) | | (1.15) | (1.15) |
| tablets | sorbitan | | | | | | |
| | monooleate | | | | | | |
| | (TWEEN-80) | | | | | | |
| 2 × 200 mg | Sodium lauryl | 36.9 (31.4) | 1.98 | 17.6 | 2.84 | 1.00 | 0 |
| Enteric-coated | sulfate | | | (16.2) | | (0.00) | |
| tablets | | | | | | | |
| 2 × 200 mg | Sodium | 46.7 (28.9) | 2.51 | 24.8 | 4.00 | 1.67 | 0.17 |
| Enteric-coated | caprate | | | (20.4) | | (1.26) | (0.29) |
| tablets | | | | | | | |
| 2 × 200 mg | EDTA | 24.6 (14.1) | 1.32 | 13.5 | 2.18 | 2.17 | 1.18 |
| Enteric-coated | | | | (9.99) | | (1.61) | (0.75) |
| tablets | | | | | | | |
| 2 × 200 mg | Palmitoyl | 29.5 (25.9) | 1.59 | 15.5 | 2.51 | 2.50 | 1.17 |
| Enteric-coated | carnitine | | | (16.4) | | (1.32) | (2.02) |
| tablets | | | | | | | |

^{a}Dose-normalized ratios of means to immediate release capsules

### Example 11

### Pharmacokinetic Study of Single Dose of 2-(8,9-dioxo-2,6-diazabicyclo[5.2.0]non-1(7)-en-2-yl)ethyl]phosphonic Acid

A study was conducted as an ascending single-dose tolerance study with fasting administration of 500, 1000, 2000, and 4000 mg oral doses. In each cohort, 8 subjects received either placebo (2 subjects) or the prescribed dose of 2-(8,9-dioxo-2,6-diazabicyclo[5.2.0]non-1(7)-en-2-yl)ethyl]phosphonic acid (6 subjects). Subjects in the fasting 1000-mg cohort were crossed over to receive a 1000-mg postprandial dose in study period 2. Additionally, the 2000-mg dose level was repeated in a cohort of elderly subjects.

Table 4 summarizes the pharmacokinetic profile of 2-(8,9-dioxo-2,6-diazabicyclo[5.2.0]non-1(7)-en-2-yl)ethyl]phosphonic acid capsules from all cohorts in the study following oral administration in the fasting state. 2-(8,9-Dioxo-2,6-diazabicyclo[5.2.0]non-1(7)-en-2-yl)ethyl]phosphonic acid was rapidly absorbed, attaining peak plasma concentrations within 1 to 2 hours after administration. 2-(8,9-Dioxo-2,6-diazabicyclo[5.2.0]non-1(7)-en-2-yl)ethyl]phosphonic acid plasma concentrations subsequently declined with a mono- or occasionally bi-exponential elimination with a mean t_{1/2} of 6 to 16 hours, but the estimates of t_{1/2} were not always reliable. For the subjects in the first cohort, the mean absolute bioavailability was estimated to be 4.3%.

For the subjects in the second cohort, administration of 2-(8,9-dioxo-2,6-diazabicyclo[5.2.0]non-1(7)-en-2-yl)ethyl]phosphonic acid following a standardized high-fat, high-calorie meal slowed the adsorption of 2-(8,9-dioxo-2,6-diazabicyclo[5.2.0]non-1(7)-en-2-yl)ethyl]phosphonic acid, prolonging the mean Tₘₐₓ by about 2 hours (from 0.88 to 2.92 hours) and lowering the mean Cₘₐₓ by 67% (from 1179 to 392 ng/mL). Additionally, administration with food reduced the mean 2-(8,9-dioxo-2,6-diazabicyclo[5.2.0]non-1(7)-en-2-yl)ethyl]phosphonic acid AUC by 57% (from 5132 to 2210 ng·h/mL).

In elderly subjects receiving 2-(8,9-dioxo-2,6-diazabicyclo[5.2.0]non-1(7)-en-2-yl)ethyl]phosphonic acid 2000 mg (fasting, single-dose), the mean oral-dose clearance (C1/F) was approximately 10% lower than that for the healthy adult subjects receiving the same dose (3.14 versus 3.50 L/h/kg). Consequently, the mean 2-(8,9-dioxo-2,6-diazabicyclo[5.2.0]non-1(7)-en-2-yl)ethyl]phosphonic acid AUC was slightly higher in the elderly subjects (8891 versus 7644 ng·h/mL).

TABLE 4
Mean ± SD (% CV) of 2-(8,9-dioxo-2,6-diazabicyclo[5.2.0]non-
1(7)-en-2-yl)ethyl]phosphonic acid PK parameters after a single
administration in healthy subjects (n = 6 per cohort)

| | Cmax | Tmax | T½ | AUC |
|---|---|---|---|---|
| Dose | (ng/mL) | (h) | (h) | (ng · h/mL) |
| 500 mg | 320 ± 63 | 1.9 ± 0.7 | 6.7 ± 6.4 | 2140 ± 542 |
| Oral, fasting | (20%) | (39%) | (96%) | (25%) |
| 100 mg | 4007 ± 512 | 0.95 ± 0.11 | 8.3 ± 8.8 | 10226 ± 1719 |
| 1-hour, | (13%) | (12%) | (106%) | (17%) |
| infusion | | | | |
| 1000 mg | 1179 ± 618 | 0.88 ± 0.59 | 14.6 ± 9.3 | 5132 ± 1420 |
| Oral, fasting | (52%) | (67%) | (64%) | (28%) |
| 1000 mg | 392 ± 236 | 2.92 ± 1.20 | 16.0 ± 10.0 | 2210 ± 578 |
| Oral, fed | (60%) | (41%) | (63%) | (26%) |
| 2000 mg | 1786 ± 1364 | 1.42 ± 0.96 | 6.4 ± 2.0 | 7644 ± 1828 |
| Oral, fasting | (76%) | (68%) | (32%) | (25%) |
| 2000 mg | 1606 ± 628 | 1.25 ± 0.61 | 14.1 ± 11.1 | 8891 ± 1437 |
| Oral, fasting | (39%) | (49%) | (79%) | (16%) |
| (elderly) | | | | |
| 4000 mg | 1488 ± 436 | 2.60 ± 1.08 | 9.1 ± 4.6 | 8982 ± 1981 |
| Oral, fasting | (29%) | (42%) | (51%) | (22%) |

*AUC in Table 4 is reported for t = 0 to ∞.

### Example 12

### Pharmacokinetic Study of Multiple Dose of 2-(8,9-Dioxo-2,6-diazabicyclo[5.2.0]non-1(7)-en-2-yl)ethyl]phosphonic Acid

A study was conducted as an ascending multiple-dose tolerance study of 2-(8,9-dioxo-2,6-diazabicyclo[5.2.0]non-1(7)-en-2-yl)ethyl]phosphonic acid with administration of 200, 400, 800, and 1600 mg oral doses in healthy subjects over a 14-day period.

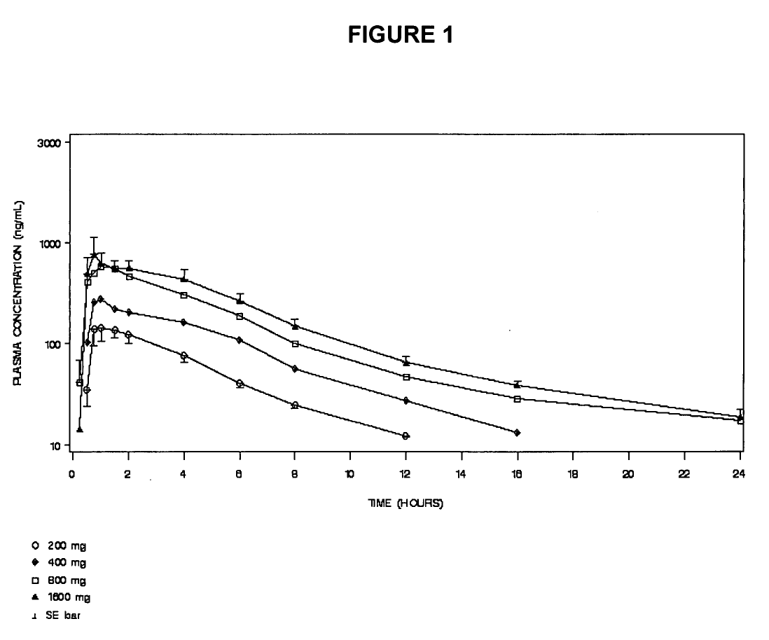

Table 5 describes the pharmacokinetic data following multiple ascending doses. FIGS. 1 to 6 show the following:

FIG. 1 is a plot of mean plasma concentration (in ng/mL) as a function of time (in hours) for a single dose of [2-(8,9-dioxo-2,6-diazabicyclo[5.2.0]non-1(7)-en-2-yl)ethyl]phosphonic acid in healthy subjects after receiving 200, 400, 800, or 1600 mg of [2-(8,9-dioxo-2,6-diazabicyclo[5.2.0]non-1(7)-en-2-yl)ethyl]phosphonic acid.

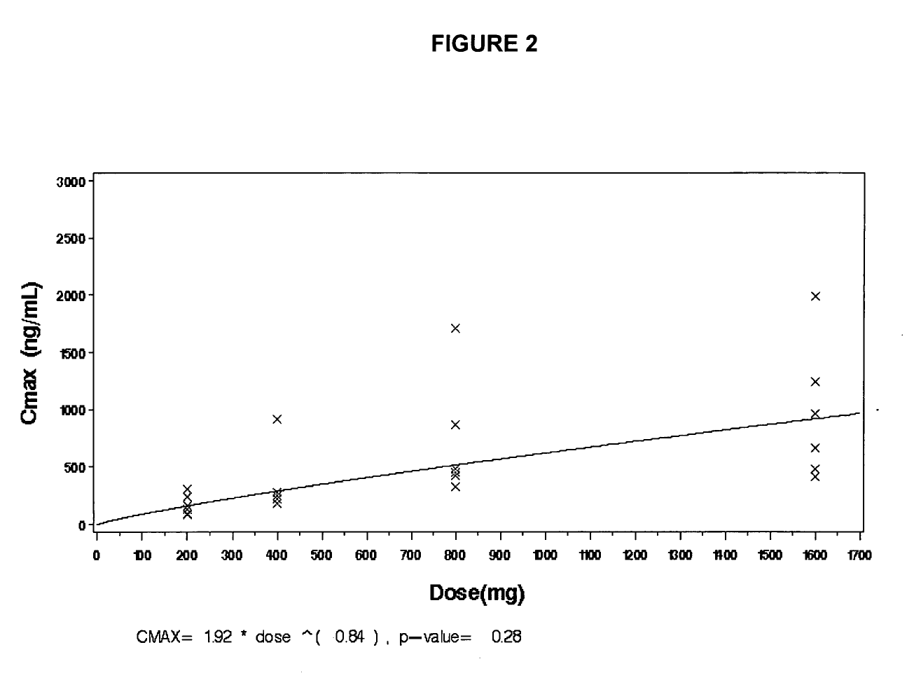

FIG. 2 is a plot of Cₘₐₓ (in ng/mL) as a function of dose (in mg) for a single dose of [2-(8,9-dioxo-2,6-diazabicyclo[5.2.0]non-1(7)-en-2-yl)ethyl]phosphonic acid in healthy subjects after receiving 200, 400, 800, or 1600 mg of [2-(8,9-dioxo-2,6-diazabicyclo[5.2.0]non-1(7)-en-2-yl)ethyl]phosphonic acid.

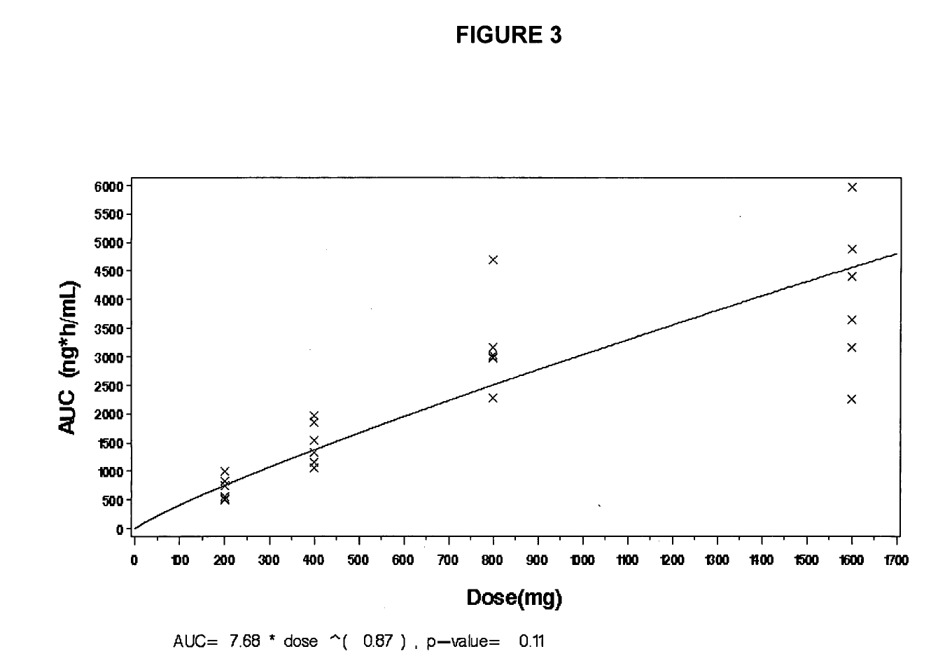

FIG. 3 is a plot of AUC (in ng·h/mL, t=0 to ∞) as a function of dose (in mg) for a single dose of [2-(8,9-dioxo-2,6-diazabicyclo[5.2.0]non-1(7)-en-2-yl)ethyl]phosphonic acid in healthy subjects after receiving 200, 400, 800, or 1600 mg of [2-(8,9-dioxo-2,6-diazabicyclo[5.2.0]non-1(7)-en-2-yl)ethyl]phosphonic acid.

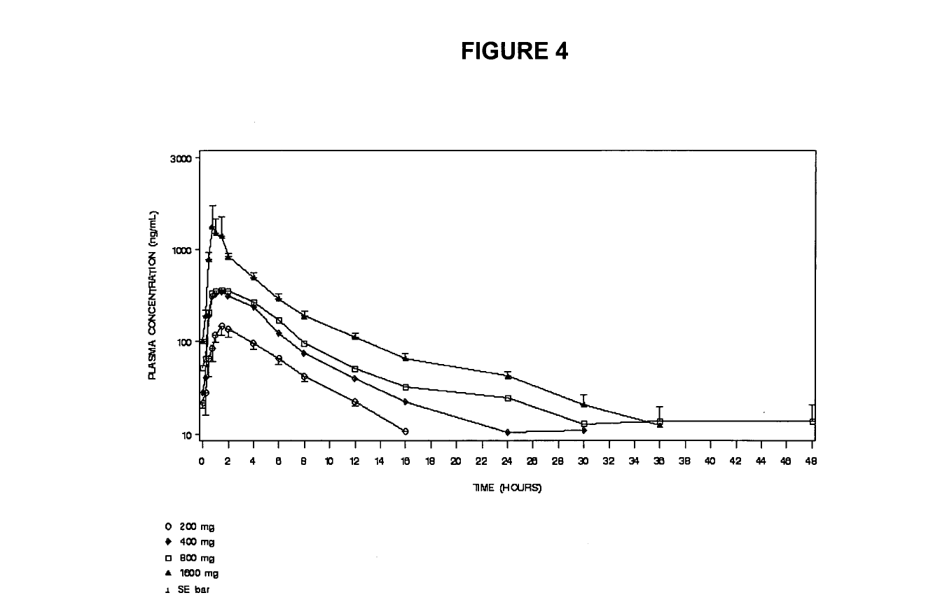

FIG. 4 is a plot of mean steady state plasma concentration (in ng/mL) as a function of time (in hours) for [2-(8,9-dioxo-2,6-diazabicyclo[5.2.0]non-1(7)-en-2-yl)ethyl]phosphonic acid in healthy subjects after receiving 200, 400, 800, or 1600 mg of [2-(8,9-dioxo-2,6-diazabicyclo[5.2.0]non-1(7)-en-2-yl)ethyl]phosphonic acid.

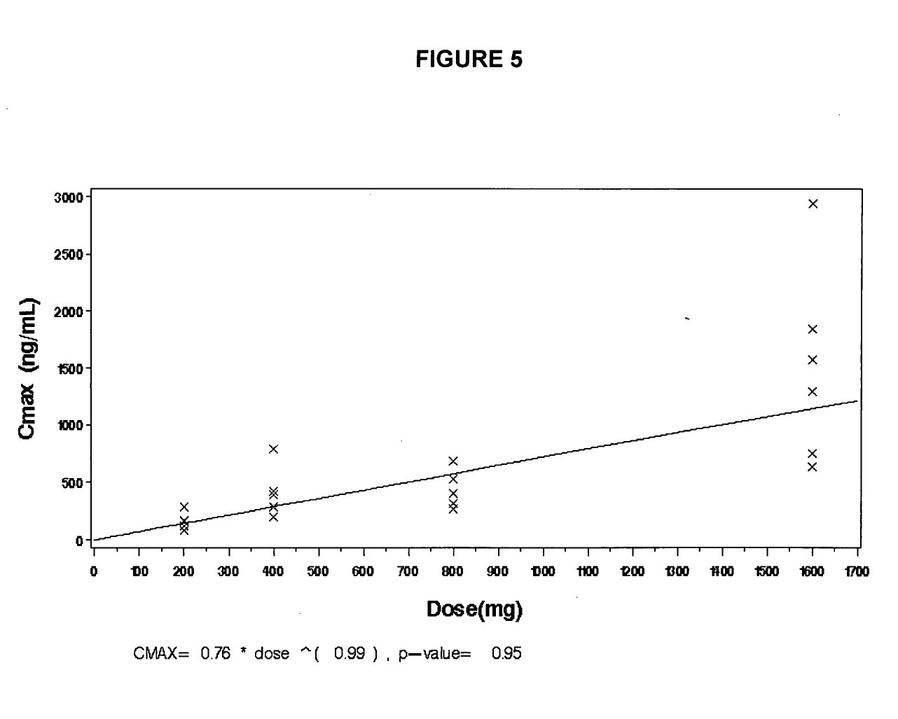

FIG. 5 is a plot of steady state Cₘₐₓ (in ng/mL) as a function of dose (in mg) for [2-(8,9-dioxo-2,6-diazabicyclo[5.2.0]non-1(7)-en-2-yl)ethyl]phosphonic acid in healthy subjects after receiving 200, 400, 800, or 1600 mg of [2-(8,9-dioxo-2,6-diazabicyclo[5.2.0]non-1(7)-en-2-yl)ethyl]phosphonic acid.

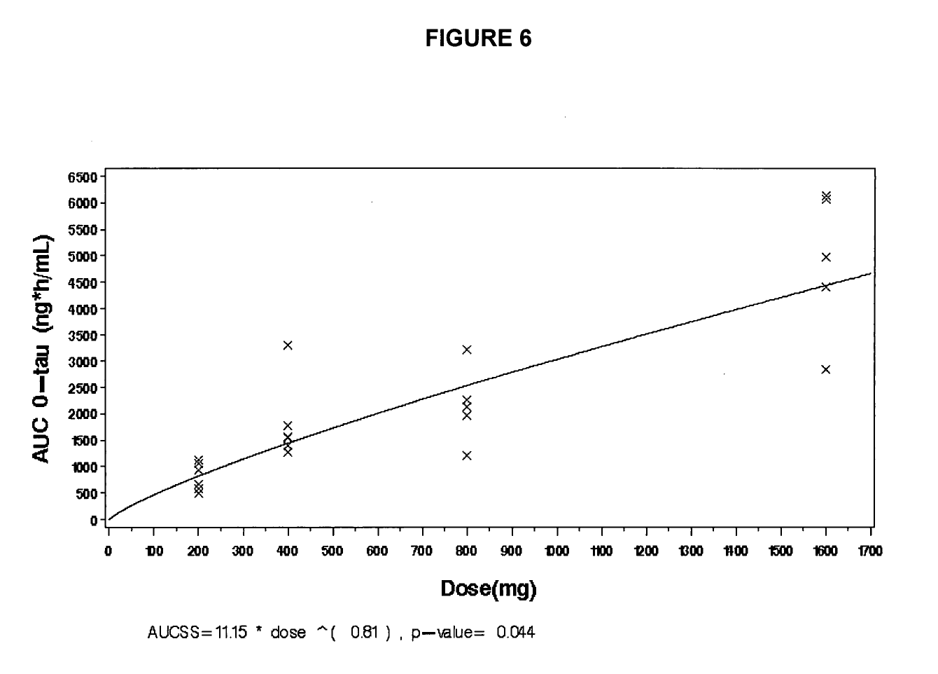

FIG. 6 is a plot of steady state AUC (in ng·h/mL) as a function of dose (in mg) for [2-(8,9-dioxo-2,6-diazabicyclo[5.2.0]non-1(7)-en-2-yl)ethyl]phosphonic acid in healthy subjects after receiving 200, 400, 800, or 1600 mg of [2-(8,9-dioxo-2,6-diazabicyclo[5.2.0]non-1(7)-en-2-yl)ethyl]phosphonic acid.

TABLE 5
Mean ± SD (% CV) of 2-(8,9-dioxo-2,6-
diazabicyclo[5.2.0]non-1(7)-en-2-yl)ethyl]phosphonic
acid PK parameters in healthy subjects (n = 6 per cohort)

| | Cmax | Tmax | T½ | AUC** |
|---|---|---|---|---|
| Dose | (ng/mL) | (h) | (h) | (ng · h/mL) |
| Single dose | | | | |
| administration | | | | |
| 200 mg | 172 ± 90 | 1.3 ± 0.5 | 3.5 ± 0.6 | 699 ± 201 |
| Oral, fasting | (52%) | (40%) | (18%) | (29%) |
| 400 mg | 346 ± 282 | 2.0 ± 1.6 | 4.5 ± 1.2 | 1487 ± 371 |
| Oral, fasting | (82%) | (79%) | (27%) | (25%) |
| 800 mg | 715 ± 524 | 1.4 ± 0.7 | 8.3 ± 1.3 | 3188 ± 800 |
| Oral, fasting | (73%) | (47%) | (16%) | (25%) |
| 1600 mg | 962 ± 592 | 1.3 ± 1.3 | 6.9 ± 1.7 | 4057 ± 1316 |
| Oral, fasting | (62%) | (98%) | (24%) | (32%) |
| Multiple dose | | | | |
| administration | | | | |
| (day 14) | | | | |
| 200 mg q 12 h | 153 ± 75 | 1.2 ± 0.3 | 6.6 ± 3.9 | 817 ± 265 |
| Oral, fed | (49%) | (27%) | (60%) | (32%) |
| 400 mg q 12 h | 398 ± 209 | 1.6 ± 1.3 | 11.9 ± 10.4 | 1811 ± 747 |
| Oral, fasting | (53%) | (83%) | (87%) | (41%) |
| 800 mg q 12 h | 436 ± 151 | 1.4 ± 0.6 | 16.9 ± 8.8 | 2175 ± 644 |
| Oral, fasting | (35%) | (40%) | (52%) | (30%) |
| 1600 mg q 12 h | 1509 ± 842 | 1.4 ± 1.3 | 10.5 ± 6.6 | 4909 ± 1211 |
| Oral, fasting | (56%) | (90%) | (63%) | (25%) |

**AUC reported for single dose administration in Table 5 is for t = 0 to ∞ AUC reported for multiple dose administration is for t = 0 to 12 hours (tau).

When ranges are used herein for physical properties, such as molecular weight, or chemical properties, such as chemical formulae, all combinations and subcombinations of ranges specific embodiments therein are intended to be included.

The disclosures of each patent, patent application and publication cited or described in this document are hereby incorporated herein by reference, in their entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A solid, pharmaceutical dosage form, comprising:
at least one compound of formula (I) or a pharmaceutically acceptable salt thereof: 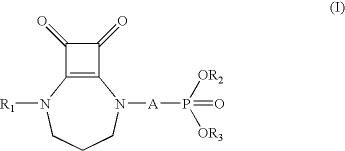
wherein:
R₁ is hydrogen, a C₁ to C₆ alkyl group, a C₂ to C₇ acyl group, a C₁ to C₆ alkanesulfonyl group, or a C₆ to C₁₄ aroyl group;
A is alkylenyl of 1 to 4 carbon atoms or alkenylenyl of 2 to 4 carbon atoms;
R₂ and R₃ are independently selected from hydrogen, 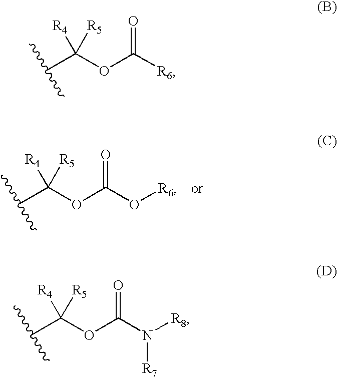
with the proviso that at least one of R₂ and R₃ is other than hydrogen;
R₄ and R₅ are independently selected from hydrogen, a C₁ to C₄ alkyl group, a C₅ to C₇ aryl group, a C₆ to C₁₅ aralkyl group having 5 to 7 carbon atoms in the aryl ring, a C₂ to C₇ alkenyl group, or C₂ to C₇ alkynyl group, or R₄ and R₅ may together form a spiro C₃ to C₈ carbocyclic ring;
R₆ is a C₁ to C₁₂ linear or branched alkyl group, a C₂ to C₇ linear or branched alkenyl or alkynyl group, a C₅ to C₁₃ aryl group, a C₆ to C₂, aralkyl group having 5 to 13 carbon atoms in the aryl moiety; a 5 to 13 membered heteroaryl group, a 6 to 21 membered heteroaralkyl group having 5 to 13 members in the heteroaryl moiety, a C₄ to C₈ cycloalkyl group, a C₅ to C₁₆ cycloalkylalkyl group having 4 to 8 carbon atoms in the cycloalkyl ring;
R₇ and R₈ are independently selected from hydrogen, a C₁ to C₁₂ linear or branched alkyl group, a C₂ to C₇ linear or branched alkenyl or alkynyl group, a C₅ to C₁₃ aryl group, a C₆ to C₂, aralkyl group having 5 to 13 carbon atoms in the aryl moiety, a 5 to 13 membered heteroaryl group, a 6- to 21-membered heteroaralkyl group having 5 to 13 members in the heteroaryl moiety, or R₇ and R₈ may together form a cycloalkyl or heterocycloalkyl group having in the ring 4 to 8 carbon atoms and optionally one to two atoms selected from nitrogen, oxygen or sulfur;
wherein any R₁ to R₈ group having an aryl, heteroaryl, cycloalkyl or heterocycloalkyl moiety may optionally be substituted on the aryl, heteroaryl, cycloalkyl or heterocycloalkyl moiety with 1 to about 5 substituents independently selected from a halo, a cyano, nitro or hydroxyl group, a C₁ to C₆ alkyl group, or a C₁ to C₆ alkoxy group; and
at least one pharmaceutically acceptable absorption enhancer.

2. A solid, pharmaceutical dosage form, comprising:
at least one compound of formula (I) or a pharmaceutically acceptable salt thereof: 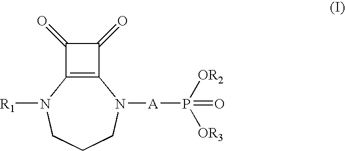
wherein:
R₁ is hydrogen;
A is —(CH₂)ₙ—, where n is 2; and
R₂ and R₃ are hydrogen; and
at least one pharmaceutically acceptable absorption enhancer.

3. A dosage form according to claim 1 or 2 that is a powder, a capsule or a tablet.

4. A dosage form according to claim 1 or 2 that is an enteric-coated capsule or an enteric-coated tablet.

5. A dosage form according to claim 4 that comprises an anionic polymer selected from the group consisting of a methacrylic acid copolymer, cellulose acetate phthalate, hydroxpropylmethylcellulose phthalate, polyvinyl acetate phthalate, shellac, hydroxpropylmethylcellulose acetate succinate, and carboxy-methylcellulose.

6. A dosage form according to claim 1 or 2 that is an immediate release capsule or an immediate release tablet.

7. A dosage form according to claim 1 or 2 comprising about 25% by weight to about 99.5% by weight, based on the total weight of said dosage form, of said compound of formula (I).

8. A dosage form according to claim 7 comprising about 50% by weight to about 99.5% by weight, based on the total weight of said dosage form, of said compound of formula (I).

9. A dosage form according to claim 8 comprising about 60% by weight to about 99.5% by weight, based on the total weight of said dosage form, of said compound of formula (I).

10. A dosage form according to claim 9 comprising about 70% by weight to about 99.5% by weight, based on the total weight of said dosage form, of said compound of formula (I).

11. A dosage form according to claim 1, wherein R₁ is H or a C₁ to C₄ alkyl group.

12. A dosage form according to claim 1, wherein A is an alkylenyl group having the formula —(CH₂)ₙ—, where n is 1 to 3.

13. A dosage form according to claim 1, wherein R₂ is H.

14. A dosage form according to claim 1, wherein R₄ and R₅ are independently H or a C₁ to C₄ alkyl group, and R₆ is a C₃ to C₁₀ linear or branched alkyl group, a C₅ to C₇ aryl group, a 5- to 7-membered heteroaryl group, or a cycloalkyl group having in the ring 5 to 7 carbon atoms.

15. A dosage form according to claim 14, wherein R₆ is a C₅ to C₇ aryl group.

16. A dosage form according to claim 1, wherein R₂ and R₃ are both hydrogen.

17. A dosage form according to claim 1, wherein at least one of said compounds of formula (I) is:
3-{2-[8,9-dioxo-2,6-diazabicyclo[5.2.0]non-1(7)-en-2-yl]ethyl}-3-oxido-7-oxo-7-phenyl-2,4,6-trioxa-3-phosphahept-1-yl benzoate;
3-{2-[8,9-dioxo-2,6-diazabicyclo[5.2.0]non-1(7)-en-2-yl]ethyl}-3-oxido-7-oxo-8-propyl-2,4,6-trioxa-3-phosphaundec⁻¹-yl 2-propylpentanoate;
2,2-dimethyl-propionic acid (2,2-dimethyl-propionyloxymethoxy)-[2-(8,9-dioxo-2,6-diaza-bicyclo[5.2.0]non-1(7)-en-2-yl)-ethyl]-phosphinoyloxymethyl ester;
7-cyclohexyl-3-{2-[8,9-dioxo-2,6-diazabicyclo[5.2.0]non-1(7)-en-2-yl]ethyl}-1,5-dimethyl-3-oxido-7-oxo-2,4,6-trioxa-3-phosphahept-1-yl cyclohexanecarboxylate;
7-cyclohexyl-3-{2-[8,9-dioxo-2,6-diazabicyclo[5.2.0]non-1(7)-en-2-yl]ethyl}-3-oxido-7-oxo-2,4,6-trioxa-3-phosphahept-1-yl cyclohexanecarboxylate;
[2-(8,9-dioxo-2,6-diaza-bicyclo[5.2.0]non-1-(7)-en-2-yl)-ethyl]-phosphonic acid diisopropoxycarbonyl oxymethyl ester;
[2-[8,9-dioxo-2,6-diazabicyclo[5.2.0]non-1(7)-en-2-yl]ethyl]-phosphonic acid bis [1-(benzoyloxy)ethyl] ester;
benzoic acid [2-(8,9-dioxo-2,6-diaza-bicyclo[5.2.0]non-1(7)-en-2-yl)-ethyl]-hydroxy-phosphinoyloxymethyl ester;
[2-(8,9-dioxo-2,6-diaza-bicyclo[5.2.0]non-1(7)-en-2-yl)-ethyl]-phosphonic acid di-dimethylcarbamoyloxymethyl ester;
or a pharmaceutically acceptable salt thereof.

18. A dosage form according to claim 1 comprising about 0.25 weight % to about 50 weight %, based on the total weight of said dosage form, of said absorption enhancer.

19. A dosage form according to claim 1 or 2, wherein said pharmaceutically acceptable absorption enhancer is surfactant, bile salt, fatty acid, fatty acid salt, chelating agent, acyl carnitine, acyl choline, or a mixture thereof.

20. A dosage form according to claim 19, wherein said surfactant is ionic surfactant, nonionic surfactant, or a mixture thereof.

21. A dosage form according to claim 20, wherein said ionic surfactant is sodium lauryl sulfate, dioctyl sodium sulfosuccinate, or a mixture thereof.

22. A dosage form according to claim 20, wherein said nonionic surfactant is polyoxyethylene alkyl ether, polyoxyethylene alkyl ester, polysorbate, or a mixture thereof.

23. A dosage form according to claim 22, wherein said polyoxyethylene alkyl ester is polyethylene glycol-20 sorbitan monooleate.

24. A dosage form according to claim 19, wherein said bile salt is sodium cholate, sodium deoxycholate, or a mixture thereof.

25. A dosage form according to claim 19, wherein said fatty acid is oleic acid.

26. A dosage form according to claim 19, wherein said fatty acid salt is sodium caprate.

27. A dosage form according to claim 19, wherein said chelating agent is ethylenediaminetetraacetic acid.

28. A dosage form according to claim 19, wherein said acyl carnitine is palmitoyl carnitine.

29. A dosage form according to claim 19, wherein said acyl choline is lauroyl choline.

30. A dosage form according to claim 1 or 2, further comprising a filler, disintegrant, binder, lubricant, or a mixture thereof.

31. A dosage form according to claim 30, wherein said filler is lactose, microcrystalline cellulose, mannitol, calcium phosphate, pregelatinized starch, pregelatinized sucrose, or a mixture thereof.

32. A dosage form according to claim 30, wherein said disintegrant is croscarmellose sodium, starch, sodium starch glycolate, pregelatinized starch, crospovidone, or a mixture thereof.

33. A dosage form according to claim 30, wherein said binder is povidone, hydroxypropylmethylcellulose, gelatin, gum, or a mixture thereof.

34. A dosage form according to claim 30, wherein said lubricant is magnesium stearate, sodium stearyl fumarate, or a mixture thereof.

35. A single dosage form, comprising the dosage form according to claim 1 or 2.

36. A multiple dosage form, comprising the dosage form according to claim 1 or 2.

37. A method for treating at least one condition in a mammal selected from a cerebral vascular disorder selected from cerebral ischemia, cerebral infarction or cerebral vasospasm; cerebral trauma; muscular spasm; a convulsive disorder selected from epilepsy or status epilepticus; hypoglycemia; cardiac arrest; asphyxia anoxia; or spinal chord injury, comprising the step of:
administering orally to a mammal in need thereof an effective amount of the dosage form according to claim 1 or 2.

38. A method for treating at least one condition in a mammal selected from glaucoma or diabetic end organ complications, comprising the step of:
administering orally to a mammal in need thereof an effective amount of the dosage form according to claim 1 or 2.

39. A method for treating at least one condition in a mammal selected from anxiety disorders; mood disorders; schizophrenia; schizophreniform disorder; or schizoaffective disorder, comprising the step of:
administering orally to a mammal in need thereof an effective amount of the dosage form according to claim 1 or 2.

40. A method as claimed in claim 39, wherein the anxiety disorder is selected from panic attack, agoraphobia, panic disorder, specific phobia, social phobia, obsessive compulsive disorder, posttraumatic stress disorder, acute stress disorder, generalized anxiety disorder, separation anxiety disorder, or substance-induced anxiety disorder; or the mood disorder is selected from bipolar disorders, depressive disorders selected from major depressive disorder, dysthymic disorder, or substance-induced mood disorder, or mood episodes selected from major depressive episode, manic episode, mixed episode, or hypomanic episode.

41. A method for treating at least one neurodegenerative disorder in a mammal selected from Huntingdon's disease, Alzheimer's disease, amyotrophic lateral sclerosis, chronic dementia, or cognitive impairment, comprising the step of:
administering orally to a mammal in need thereof an effective amount of the dosage form according to claim 1 or 2.

42. A method for treating Parkinson's disease, comprising the step of:
administering orally to a mammal in need thereof an effective amount of the dosage form according to claim 1 or 2.

43. A method for treating at least one condition in a mammal selected from inflammatory diseases; fibromyalgia; complications from herpes zoster; prevention of tolerance to opiate analgesia; or withdrawal symptoms from addictive drugs, comprising the step of:
administering orally to a mammal in need thereof an effective amount of the dosage form according to claim 1 or 2.

44. A method for treating pain in a mammal, comprising the step of:
administering orally to a mammal in need thereof an effective amount of the dosage form according to claim 1 or 2.

45. A method according to claim 44 further comprising administering a therapeutically effective amount of at least one pain relieving agent.

46. A method according to claim 44, wherein the pain is at least one of neuropathic pain; cancer pain; visceral pain associated with pancreatitis or abdominal, pelvic or perineal regions; musculoskeletal pain associated with lower or upper back, spine, fibromylagia, temporomandibular joint, or myofascial pain syndrome; bony pain associated with bone or joint degenerating disorders; headaches; or pain associated with infections, sickle cell anemia, autoimmune disorders, multiple sclerosis, dental procedures, burns or inflammation.

47. A method according to claim 46, wherein the pain comprises neuropathic pain and is associated with at least one of diabetic neuropathy, peripheral neuropathy, post-herpetic neuralgia, trigeminal neuralgia, lumbar or cervical radiculopathies, fibromyalgia, glossopharyngeal neuralgia, reflex sympathetic dystrophy, casualgia, thalamic syndrome, nerve root avulsion, or nerve damage cause by injury selected from phantom limb pain, reflex sympathetic dystrophy or postthoracotomy pain, cancer, chemical injury, toxins, nutritional deficiencies, or viral or bacterial infections.

48. A method according to claim 46, wherein said pain is small fiber neuropathy.

49. A method according to claim 46, wherein said pain is large fiber neuropathy.

50. A method according to claim 46, wherein said pain is peripheral neuropathy.

51. A method according to claim 46, wherein said pain is central neuropathy.

52. A method according to claim 46, wherein said pain is post herpetic neuralgia.

53. A method according to claim 46, wherein said pain is post-surgical pain.

54. A solid, immediate release pharmaceutical composition, comprising:
at least one compound of formula (I) according to claim 1 or 2 or a pharmaceutically acceptable salt thereof wherein said composition has a bulk density of at least about 0.5 g/cm³.

55. A solid, immediate release pharmaceutical composition according to claim 54, wherein said composition has a bulk density of at least about 0.8 g/cm³.

56. A solid, immediate release pharmaceutical composition according to claim 54, wherein said composition is granular.

57. A solid, immediate release pharmaceutical composition according to claim 54,
wherein said solid composition further comprises at least one binder.

58. A solid, immediate release pharmaceutical composition according to claim 57,
wherein said binder is povidone.

59. A solid, immediate release pharmaceutical composition according to claim 58,
wherein said povidone is present at a level of at least about 1.5% by weight, based on the total weight of said composition.

60. A solid, immediate release pharmaceutical composition according to claim 59,
wherein said povidone is present at a level of at least about 2.5% by weight, based on the total weight of said composition.

61. A solid, immediate release pharmaceutical composition according to claim 54,
wherein said solid composition further comprises at least one disintegrant or filler.

62. A solid, immediate release pharmaceutical composition according to claim 61,
wherein said filler is microcrystalline cellulose.

63. A solid, immediate release pharmaceutical composition according to claim 61,
wherein said disintegrant is croscarmellose sodium.

64. A solid, pharmaceutical dosage form, comprising:
the solid, immediate release pharmaceutical composition according to claim 54.

65. A dosage form according to claim 64 that is a capsule or a tablet.

66. A single dosage form, comprising the dosage form according to claim 64.

67. A multiple dosage form, comprising the dosage form according to claim 64.

68. A capsule, comprising:
granular particles comprising the solid, immediate release pharmaceutical composition according to claim 54.

69. A tablet, comprising:
granular particles comprising the solid, immediate release pharmaceutical composition according to claim 54.

70. A method for treating at least one condition in a mammal selected from a cerebral vascular disorder selected from cerebral ischemia, cerebral infarction or cerebral vasospasm; cerebral trauma; muscular spasm; a convulsive disorder selected from epilepsy or status epilepticus; hypoglycemia; cardiac arrest; asphyxia anoxia; or spinal chord injury, comprising the step of:
administering orally to a mammal in need thereof an effective amount of the solid, immediate release pharmaceutical composition according to claim 54.

71. A method for treating at least one condition in a mammal selected from glaucoma or diabetic end organ complications, comprising the step of:
administering orally to a mammal in need thereof an effective amount of the solid, immediate release pharmaceutical composition according to claim 54.

72. A method for treating at least one condition in a mammal selected from anxiety disorders; mood disorders; schizophrenia; schizophreniform disorder; or schizoaffective disorder, comprising the step of:
administering orally to a mammal in need thereof an effective amount of the solid, immediate release pharmaceutical composition according to claim 54.

73. A method as claimed in claim 72, wherein the anxiety disorder is selected from panic attack, agoraphobia, panic disorder, specific phobia, social phobia, obsessive compulsive disorder, posttraumatic stress disorder, acute stress disorder, generalized anxiety disorder, separation anxiety disorder, or substance-induced anxiety disorder; or the mood disorder is selected from bipolar disorders, depressive disorders selected from major depressive disorder, dysthymic disorder, or substance-induced mood disorder, or mood episodes selected from major depressive episode, manic episode, mixed episode, or hypomanic episode.

74. A method for treating at least one neurodegenerative disorder in a mammal selected from Huntingdon's disease, Alzheimer's disease, amyotrophic lateral sclerosis, chronic dementia, or cognitive impairment, comprising the step of:
administering orally to a mammal in need thereof an effective amount of the solid, immediate release pharmaceutical composition according to claim 54.

75. A method for treating Parkinson's disease, comprising the step of:
administering orally to a mammal in need thereof an effective amount of the solid, immediate release pharmaceutical composition according to claim 54.

76. A method for treating at least one condition in a mammal selected from inflammatory diseases; fibromyalgia; complications from herpes zoster; prevention of tolerance to opiate analgesia; or withdrawal symptoms from addictive drugs, comprising the step of:
administering orally to a mammal in need thereof an effective amount of the solid, immediate release pharmaceutical composition according to claim 54.

77. A method for treating pain in a mammal, comprising the step of:
administering orally to a mammal in need thereof an effective amount of the solid, immediate release pharmaceutical composition according to claim 54.

78. A method according to claim 77 further comprising administering a therapeutically effective amount of at least one pain relieving agent.

79. A method according to claim 77, wherein the pain is at least one of neuropathic pain; cancer pain; visceral pain associated with pancreatitis or abdominal, pelvic or perineal regions; musculoskeletal pain associated with lower or upper back, spine, fibromylagia, temporomandibular joint, or myofascial pain syndrome; bony pain associated with bone or joint degenerating disorders; headaches; or pain associated with infections, sickle cell anemia, autoimmune disorders, multiple sclerosis, dental procedures, burns or inflammation.

80. A method according to claim 79, wherein the pain comprises neuropathic pain and is associated with at least one of diabetic neuropathy, peripheral neuropathy, post-herpetic neuralgia, trigeminal neuralgia, lumbar or cervical radiculopathies, fibromyalgia, glossopharyngeal neuralgia, reflex sympathetic dystrophy, casualgia, thalamic syndrome, nerve root avulsion, or nerve damage cause by injury selected from phantom limb pain, reflex sympathetic dystrophy or postthoracotomy pain, cancer, chemical injury, toxins, nutritional deficiencies, or viral or bacterial infections.

81. A method according to claim 79, wherein said pain is small fiber neuropathy.

82. A method according to claim 79, wherein said pain is large fiber neuropathy.

83. A method according to claim 79, wherein said pain is peripheral neuropathy.

84. A method according to claim 79, wherein said pain is central neuropathy.

85. A method according to claim 79, wherein said pain is post herpetic neuralgia.

86. A method according to claim 79; wherein said pain is post-surgical pain.

87. A solid, immediate release pharmaceutical composition according to claim 54,
wherein said composition exhibits a plasma Cₘₐₓ, upon administration to a subject in need thereof, for the compound of formula (I) of about 80 ng/mL to about 4200 ng/mL.

88. A solid, immediate release pharmaceutical composition according to claim 54,
wherein said composition exhibits a plasma Tₘₐₓ, upon administration to a subject in need thereof, for the compound of formula (I) of about 0.5 hours to about 4.0 hours.

89. A solid, immediate release pharmaceutical composition according to claim 54,
wherein said composition exhibits an AUC_{t=0 to 12 hours} upon administration to a subject in need thereof, for the compound of formula (I) of about 250 ng·h/mL to about 6000 ng·h/mL.

90. A solid, immediate release pharmaceutical composition according to claim 54,
wherein said composition is in the form of a capsule or a tablet.

91. A solid, immediate release pharmaceutical composition according to claim 90,
wherein said capsule or said tablet comprises about 200 mg to about 4000 mg of said compound of formula (I).

92. A solid, immediate release pharmaceutical composition according to claim 90,
wherein said composition is in the form a single dosage unit or multiple dosage unit.

93. A solid, immediate release pharmaceutical composition according to claim 92,
wherein said single dosage unit or said multiple dosage unit comprises from about 200 mg of said compound of formula (I) or a pharmaceutically acceptable salt thereof to about 4000 mg of said compound of formula (I) or a pharmaceutically acceptable salt thereof.

94. A solid, immediate release pharmaceutical composition according to claim 93,
wherein said single dosage unit or said multiple dosage unit comprises at least about 400 mg of said compound of formula (I) or a pharmaceutically acceptable salt thereof.

95. A solid, immediate release pharmaceutical composition according to claim 94,
wherein said single dosage unit or said multiple dosage unit comprises at least about 600 mg of said compound of formula (I) or a pharmaceutically acceptable salt thereof.

96. A process, comprising the steps of:
forming a wet granulation comprising:
at least one binder;
optionally at least one filler;
optionally at least one disintegrant; and
at least one compound of formula (I) according to claim 1 or 2, or a pharmaceutically acceptable salt thereof; and
forming a solid dosage form.

97. A process according to claim 96,
wherein said wet granulation is formed by:
dry blending at least one filler or disintegrant with said compound of formula (I) or a pharmaceutically acceptable salt thereof; and
granulating said dry blend with a solution of at least one binder to form a wet granulation.

98. A process according to claim 96,
wherein said binder is povidone.

99. A process according to claim 96,
wherein said filler is microcrystalline cellulose.

100. A process according to claim 96,
wherein said disintegrant is croscarmellose sodium.

101. A process according to claim 96,
further comprising the steps of:
drying said wet granulation;
milling said dried granulation; and
optionally blending said milled, dried granulation with one or more extragranulation components.

102. A process according to claim 96,
wherein said solid dosage form is a tablet.

103. A process according to claim 96,
wherein said solid dosage form is a capsule.

104. A product produced by the process of claim 96.

105. An immediate release solid pharmaceutical composition in single dosage unit or multiple dosage unit form, comprising:
[2-(8,9-dioxo-2,6-diazabicyclo[5.2.0]non-1(7)-en-2-yl)ethyl]phosphonic acid or a pharmaceutically acceptable salt thereof;
wherein said composition exhibits a plasma Cₘₐₓ, upon administration to a subject in need thereof, for the compound of formula (I) of about 80 ng/mL to about 4200 ng/mL.

106. An immediate release solid pharmaceutical composition in single dosage unit or multiple dosage unit form, comprising:
[2-(8,9-dioxo-2,6-diazabicyclo[5.2.0]non-1(7)-en-2-yl)ethyl]phosphonic acid or a pharmaceutically acceptable salt thereof;
wherein said composition exhibits an AUC_{t=0 to 12 hours} upon administration to a subject in need thereof, for the compound of formula (I) of about 250 ng·h/mL to about 6000 ng·h/mL.

107. A method for treating pain in a mammal, comprising the step of:
administering orally to a mammal in need thereof [2-(8,9-dioxo-2,6-diazabicyclo[5.2.0]non-1(7)-en-2-yl)ethyl]phosphonic acid or a pharmaceutically acceptable salt thereof in an amount to provide a plasma Cₘₐₓ, of about 80 ng/mL to about 4200 ng/mL of the [2-(8,9-dioxo-2,6-diazabicyclo[5.2.0]non-1(7)-en-2-yl)ethyl]phosphonic acid.

108. A method for treating pain in a mammal, comprising the step of:
administering orally to a mammal in need thereof [2-(8,9-dioxo-2,6-diazabicyclo[5.2.0]non-1(7)-en-2-yl)ethyl]phosphonic acid or a pharmaceutically acceptable salt thereof in an amount to provide an AUC_{t=0 to 12 hours} of about 250 ng·h/mL to about 6000 ng·h/mL of the [2-(8,9-dioxo-2,6-diazabicyclo[5.2.0]non-1(7)-en-2-yl)ethyl]phosphonic acid.
